(19) United States
(12) Patent Application Publication
(10) Pub. No.: US 2023/0041689 A1
(43) Pub. Date: Feb. 9, 2023

(54) COMPOUNDS AND METHODS FOR THE DETECTION OF FABRY DISEASE

(71) Applicant: JULIUS-MAXIMILIANS-UNIVERSITÄT WÜRZBURG, Würzburg (DE)

(72) Inventor: Nurcan ÜÇEYLER, Würzburg (DE)

(21) Appl. No.: 16/971,705

(22) PCT Filed: Feb. 25, 2019
(86) PCT No.: PCT/EP2019/054564

§ 371 Date: Aug. 21, 2020

(30) Foreign Application Priority Data
Feb. 23, 2018 (EP) 18000184.4

(51) Int. Cl. G01N 33/68

(52) U.S. Cl. CPC G01N 33/6893; G01N 2333/94; G01N 2405/10; G01N 2800/04; G01N 2800/52

(57) ABSTRACT

The present invention provides for compounds and methods for the detection and follow-up of Fabry disease (FD). In particular, the present invention relates to a method for detecting or diagnosing FD in a subject, comprising detecting globotriaosylceramide (Gb3) deposits in biomaterial obtained from said subject. The present invention also provides for a method for treatment monitoring of FD in a subject. Further, the present invention relates to the use of a Gb3-specific natural ligand for the detection of Gb3 deposits in biomaterial. Also provided is a kit for detecting Gb3 deposits in biomaterial obtained from a subject.

### FIELD OF THE INVENTION

The present invention relates to compounds and methods for the diagnosis and treatment monitoring of Fabry disease (FD) based on the detection of globotriaosylceramide (Gb3) deposits in biomaterial. Further provided is a kit for the detection/diagnosis or treatment monitoring or prognosis of FD.

### BACKGROUND OF THE INVENTION

FD is an X-linked lysosomal storage disorder that leads to an impairment or complete loss of function of the α-galactosidase A (α-GAL). The disease is caused by mutations in the encoding gene with subsequent lysosomal deposition of glycosphingolipids, particularly of Gb3. Intracellular Gb3 accumulation leads to functional impairment mainly of the heart kidneys, and the central and peripheral nervous system, making FD a life limiting, multiorgan disorder (Üçyler and Sommer, 2012 Schmerz. 26, 609-19) Additionally, FD is characterized by a unique pain phenotype which already manifests in early childhood and hardly responds to treatment

Epidemiological data on the incidence of FD are rare and conflicting. According to screening data of newborn boys, FD incidence reaches 1:8454 in Illinois (Burton et al., 2017 J. Pediatr. 190, 130.135) and 1:4004 in Mexico (Navarrete-Martinez et al., 2017 Mol Genet Metab. 121, 16-21). Even higher prevalence data were reported from Italy (1:3.100) and Taiwan (1:1.500) (Germain, 2010 Orphanet J Rare Dis. 5, 30).

The gene encoding the α-GAL (GAL) is located on the long arm of the X-chromosome (Xq22) and consists of seven exons. Meanwhile several hundred diverse mutations have been described (https://lih16.u.hpc.mssm.edu/pipeline/is/dbFabry/Mutation.html#), which are mostly “private” mutations in terms of being present only in members of one single family. Due to X-linked inheritance, hemizygote men are always affected, however, women may also reach every degree of disease severity.

The clinical presentation is dominated by the pattern and degree of organ involvement starting already from early childhood. Genotypes leading to a likely classical phenotype (i.e. the mutation is known to be associated with typical symptoms and signs of FD) are distinguished form those with likely non-classical phenotype (i.e. the mutation is associated with late onset or predominant involvement of one organ) (Van der Tol et al., 2015 Mol Genet Metab 114). One first symptom is pain, which is mostly triggered by heat, fever, or physical activity and which manifests as episodic pain (including pain attacks, pain crisis, allodynia or hyperalgesia) and chronic permanent pain. Furthermore, patients may develop nephropathy, spanning a spectrum from mid chronic kidney disease to renal failure and cardiomyopathy with cardiac fibrosis and arrhythmias (Üçyler and Sommer, 2012 Schmerz. 26,609-19). In the central nervous system (CNS), FD manifests with cerebral ischemic stroke particularly at young age and with microangiopathy. In the peripheral nervous system (PNS), patients typically develop small fiber neuropathy and sometimes also polyneuropathy.

Due to the diversity and variability of symptoms, the diagnosis of FD is often delayed. To make the diagnosis, biochemical methods are available that allow measuring the α-GAL activity in leucocytes besides genetic testing which as also described herein. Both methods can be performed exclusively in specialized laboratories, are expensive, and the results of the analysis are available with latencies. However, timely diagnosis is essential to start treatment before the onset of irreversible organ damage. When a patient was diagnosed with FD, a family screening should follow to identify potential further patients.

Since 2001, intravenous enzyme replacement therapy (ERT) is available with agalsidase-alpha and agalsidase-beta (Eng et al., 2001 Am J Hum Genet 68, 711-22; Schiffmann et al. 2001 JAMA 285, 2743-9). In 2016, the first oral chaperone migalestat was approved for patients carrying distinct missense mutations (Germain et al., 2016 N Engl J Med 375, 645-55). Further drugs with different mechanisms of action (e.g. inhibitors of Gb3 synthesis) are currently tested in clinical studies. Hence further drugs are expected to be licensed within the next years. ERT is a lifelong treatment which needs biweekly repetition and which is associated with immense costs. The same is true for the lately approved oral chaperone therapy with migalastat, a drug that needs to be ingested every second day. Treatment response varies between individual patients. Autoantibodies against the infused enzyme can significantly influence drug efficacy (Lenders et al., 2016 J Am Soc Nephrol 27, 256-64). So far however, there is no biomarker available that would allow the objective follow-up of FD patient's treatment response or enable patient stratification for treatment initiation.

To make the diagnosis of FD the following current diagnostic concepts/methods are used: 1) determination of the α-GAL enzyme activity in leucocytes, 2) molecular genetic analysis of the encoding GAL gene, and 3) organ biopsy (heart or kidneys) for the electron microscopic detection of Gb3 depositions.

However, there are several limitations and problems of the current diagnostic tools. α-GAL enzyme activity in leucocytes, α-GAL enzyme activity is determined in specialized laboratories using blood samples collected in ethylene diamine tetra-acetic acid (EDTA) containing monovettes. Enzyme activity can alternatively be assessed on dried blood spot cards. However, when detecting a low enzymatic activity, the repetition of the test is recommended using the conventional method with blood samples in EDTA-containing tubes, which is associated with diagnostic delay. Normal α-GAL activity virtually excludes FD In men. However, if enzyme activity is reduced, genetic testing is necessary to determine the underlying mutation, in women, α-GAL enzyme activity may be normal in up to 30% even in the presence of a pathogenic mutation. Therefore, genetic testing is mandatory in female FD patients. The results of the analysis are mostly available within 2-3 weeks.

Further, molecular genetic testing in specialized laboratories is expensive and time consuming. Analysis may result in the detection of known causative exon mutations, however, may also give equivocal data. These are polymorphisms in GAL-exons and introns and other so far unknown genetic variants (Schiffman et al. 2016 Genet Med. 18(12):1181-1185). It is not known, if such genetic findings are of biological and pathophysiological relevance, thus, their clinical consequences remain unclear. This hampers diagnostics particularly in patients with atypical symptom presentation. The crucial question, it a (very time consuming and expensive) ERT or oral treatment should be started cannot be answered in these cases. This diagnostic uncertainty causes an enormous burden on the respective patients and their relatives, who remain in the dilemma of having symptoms together with a genetic finding of uncertain significance potentially indicating an inherited fatal disease.

When the diagnosis cannot be confirmed by determining the α-GAL enzyme activity and molecular genetic analysis, tissue biopsy for electron microscopic investigation is mandatory to search for Gb3 deposits as an unequivocal proof of FD. Heart and kidneys are the organs of choice since they are most frequently involved in FD. Even if the risk for adverse effects is estimated low during such an organ biopsy, it is still an invasive method with potentially life threatening complications. For the subsequent histological assessment of the collected material, elaborated techniques such as electron microscopy are necessary, which are available only at specialized centers. These investigations are time consuming, expensive, and depend on high technical expertise. Furthermore, myocardial or kidney biopsies require hospitalization with considerable indirect medical costs and costs associated with absenteeism from work. The entire procedure including the histological analysis is completed within weeks and also the results of the electron microscopic assessment may be false positive or false negative. The detection of Gb3 deposits in human biomaterial is otherwise not included in the current diagnostic guidelines of FD.

In sum, the current state of the art does not provide a way of simply and reliably detecting Gb3 deposits in easily available biomaterial as a diagnostic marker of FD. Recently, Gb3 deposits were detected in skin punch biopsy specimens of patients with FD using a commercial antibody against Gb3 (Ûçeyler et al., 2016 Plos One). However, the method suffers from several weaknesses and requires an invasive intervention (skin punch biopsy). Furthermore the commercial antibody used in said study did not give entirely sufficient results in human skin samples. Also, FD patients often have contraindications against biopsies due to the co-medication taken (e.g. anticoagulants) or refuse repetitive invasive diagnostic procedures that might be needed for follow-up examinations.

Accordingly, there is a need in the way to find a suitable biomaterial that can be easily and repetitively obtained, and that is well accessible to Gb3 binding reagents, such as antibodies. Further, there is a demand to find specific antibodies or natural Gb3 ligands that unequivocally detect Gb3 in the respective easy available biomaterial. In sum, there is the requirement to establish a method that is easy to perform in diagnosis/detection or treatment monitoring or prognosis of FD without the necessity of demanding technology or expertise. Preferably, there should be the possibility to transfer the methods to commercially available and easy-to-use test kits, which are also suitable for self-administration.

The technical problem underlying the present application is thus to comply with these needs. The technical problem is solved by providing the embodiments reflected in the claims, described in the description and illustrated in the examples and figures that follow.

### SUMMARY OF THE INVENTION

The present invention is based at least party on the surprising finding that easily and repetitively obtainable biomaterial such as a blood smear prepared from whole blood, peripheral blood mononuclear cells (PBMCs) and epithelial cells, in particular buccal epithelial cells, provide suitable biomaterial for unequivocal detection of Gb3 deposits. In particular, these biomaterials are well suited for diagnosing/detecting FD in a subject, preferably a human subject, or treatment monitoring of FD in a patient. Furthermore, the methods of the present invention are based on non-invasive techniques which are easy to perform, inexpensive and easily transferrable to a kit for (self-) administration by physicians dealing with adult and pediatric Fabry patients, such as general practitioners, cardiologists, nephrologists, and neurologists. The present invention therefore provides valuable diagnostic tools for FD detection and treatment monitoring which overcome disadvantages of previously used methods.

Accordingly, in one aspect the present invention relates to a method for detecting or diagnosing FD in a subject, comprising detecting Gb3 deposits in biomaterial obtained from said subject, wherein said biomaterial is selected from the group consisting of (i) blood smear prepared from whole blood, (i) PBMCs. and (iii) epithelial cells. It is envisaged that the epithelial cells are buccal epithelial cells or bladder epithelial cells. Preferably, said bladder epithelial cells are present in a urine sample

According to the method for detecting or diagnosing FD, an increased amount of Gb3 deposit positive cells in said biomaterial as compared to a control is indicative for FD.

Furthermore, it is envisaged that the subject is a human subject. In some embodiments the human subject is of under 18 years of age.

The method for detecting or diagnosing FD preferably comprises: (i) depositing the biomaterial obtained from a subject to a solid support, thereby immobilizing said biomaterial, and (ii) detecting Gb3 deposit positive cells in said biomaterial. In this respect, it is envisaged that detecting Gb3 deposits positive cells in said biomaterial comprise optical visualization or chemoelectric detection of Gb3 deposits.

According to the method for detecting or diagnosing FD it is also envisaged that the method comprises smearing the biomaterial of step (a) on said support. In some embodiments said solid support is a glass slide.

The method for detecting or diagnosing FD may further comprise contacting the immobilized biomaterial with a Gb3 binding agent or a reagent metabolizing Gb3 to a Gb3 metabolic product, thereby allowing for detection of Gb3 deposits positive cells. It is envisaged that said Gb3 binding agent is a Gb3 specific antibody or a Gb3 natural ligand. Preferably, the Gb3-specific antibody comprises a label. Said label preferably comprises a fluorescent moiety. It is envisaged that the reagent metabolizing Gb3 to a Gb3 metabolic product is alpha-galactosidase. It is further envisaged that the known Gb3 natural ligand is a shiga toxin.

Furthermore, according to the method for detecting or diagnosing FD the visualization of Gb3 deposits preferably comprises an optical detection system,

According to another aspect, the present invention relates to a method for treatment monitoring of FD in a patient, comprising comparing the amount of Gb3 deposits positive cells detected in biomaterial obtained from said patient to the amount of Gb3 deposits positive cells detected in biomaterial obtained from said patient at an earlier date, wherein said biomaterial is one of (i) blood smear prepared from whole blood (i) PBMCs and (iii) epithelial cells, wherein the comparison provides an evaluation of effect of FD treatment. It is envisaged that the epithelial cells are buccal epithelial cells or bladder epithelial cells. The bladder epithelial cells are preferably present in a urine sample.

According to the method for treatment monitoring of FD, it is envisages that a decreased amount of Gb3 deposit positive cells as compared to the amount of Gb3 deposit positive cells detected at an earlier date indicates a positive treatment effect. No change or an increased number of Gb3 deposit positive cells as compared to the amount of Gb3 deposit positive cells detected at an earlier date indicates no treatment effect.

It is envisaged that the patient of the method for treatment monitoring of FD is a human. In some embodiments the human is of under 18 years of age.

It is also envisaged that the method of treatment monitoring of FD further comprises detecting Gb3 deposit positive cells in said biomaterial. Detecting preferably comprises optical visualization or chemoelectric detection of Gb3 deposits.

According to the method of treatment monitoring of FD, said treatment may comprise a compound reducing Gb3 deposits in Gb3 positive cells or a compound reducing the production of Gb3 deposits. Said treatment is preferably an ERT, a chaperone therapy or substrate reduction therapy, or a combination thereof.

Said treatment may comprise agalsidase, migalastat, lucerastat, or a combination thereof.

In a further preferred embodiment, said treatment may comprise gene therapy.

According to the method for treatment monitoring of FD, the patient preferably carries a mutation in the α-GAL gene leading to a FD phenotype. Preferably, said mutation is a nonsense mutation.

The method for treatment monitoring of FD may further comprise the steps of: a) depositing the biomaterial obtained from a patient to a solid support thereby immobilizing said biomaterial, and b) detecting Gb3 deposit positive cells in said biomaterial. Detecting preferably comprises optical visualization or chemoelectic detection of Gb3 deposits. Detecting may further comprise smearing the biomaterial of step (a) on said solid support. In some embodiments the solid support is a glass slide.

The method of treatment monitoring of FD may further comprise contacting the immobilized biomaterial with a Gb3 binding agent or a reagent metabolizing Gb3 to a Gb3 metabolic product, thereby allowing for detection of Gb3 deposit positive cells.

In some embodiments said the Gb3 binding agent is a Gb3 specific antibody or a Gb3 natural ligand. Preferably, the Gb3-specific antibody comprises a label. Said label preferably comprises a fluorescent moiety. It is envisaged that the reagent metabolizing Gb3 to a Gb3 metabolic product is alpha-galactosidase. It is further envisaged that the known Gb3 natural ligand is a shiga toxin.

Furthermore, according to the method of treatment monitoring of FD the visualization of Gb3 deposit positive cells preferably comprises an optical detection system.

According to the method for detecting or diagnosing FD or the method for treatment monitoring of FD, the PBMCs are preferably derived from venous peripheral blood. Furthermore, the whole blood is preferably venous peripheral blood or capillary blood.

It is further envisaged that the biomaterial is permeabilized or lysed.

According to another aspect, the present invention relates to the use of a Gb3-specific natural ligand for the detection of Gb3 deposits in biomaterial. The known Gb3-specific natural ligand is preferably a shiga toxin. The biomaterial is preferably selected from the group consisting of (i) whole blood (ii) PBMCs and (ii) epithelial cells. The whole blood is preferably venous peripheral blood or capillary blood. PBMCs are preferably derived from venous peripheral blood. Epithelial cells are preferably buccal epithelia cell or bladder epithelial cells, in some embodiments said bladder epithelial cells are present in a urine sample.

In another aspect, the present invention relates to a kit comprising a) a first solid support for depositing biomaterial and b) a Gb3-binding agent or a reagent metabolizing Gb3 to a Gb3 metabolic product allowing for detection of Gb3 deposits in said biomaterial.

In this context, detecting preferably comprises optical detection or chemoelectric detection of Gb3 deposits. The biomaterial is preferably selected from the group consisting of (i) whole blood, (ii) PBMCs. and (iii) epithelial cells. The whole blood is preferably venous peripheral blood or capillary blood. The PBMCs are preferably derived from venous peripheral blood. The epithelial cells are preferably buccal epithelial cell or bladder epithelial cells. The bladder epithelial cells are preferably present in a urine sample.

According to the kit of the present invention, depositing biomaterial in step (a) further comprises smearing the biomaterial on said solid support, in some embodiments the solid support is a glass slide.

It is envisaged that the biomaterial is permeabilized or lysed.

It Is further envisages that the kit according to the invention further comprises means for obtaining said biomaterial from a subject

According to the kit of the invention, the Gb3-binding agent is preferably a Gb3-specific antibody or a Gb3-specific natural ligand. In some embodiments the Gb3-specific antibody comprises a label. Preferably the label comprises a fluorescent moiety. It is envisaged that the reagent metabolizing Gb3 to a Gb3 metabolic product is alpha-galactosidase. It is further envisaged that the known Gb3 natural ligand is a shiga toxin.

According to another aspect, the present invention also refers to a method of prognosis of FD, comprising detecting Gb3 deposits in biomaterial obtained from said subject, wherein said biomaterial is selected from the group consisting of (i) blood smear prepared from whole blood, (ii) PBMCs. and (iii) epithelial cells.

### DETAILED DESCRIPTION

In order to overcome some of the short comings of the means described so far in the prior art for diagnosing FD or treatment monitoring for FD, the inventors provide herein promising new methods and kits for detecting Gb3 deposit positive cells in easily and repetitively available biomaterial obtained from subjects or patients with FD. In particular, the inventors of the present invention surprisingly discovered that Gb3 deposits can be reliably detected in biomaterial like blood cells, in particular blood smear. PBMCs or epithelial cells, in particular buccal epithelial cells, which can be easily obtained from a subject and which are well accessible to Gb3 binding reagents such as Gb3 antibodies or known natural Gb3 ligands like shiga toxin. In addition, the inventors observed that said easily accessible biomaterials can be equally used for follow-up or treatment monitoring of FD, thereby having reliably means to control treatment efficacy during therapy, as well as in prognosis of FD. Further, the invention provides for a kit to be used in a method of detecting/diagnosing, treatment monitoring or prognosis of FD, wherein Gb3 deposits in easy accessible biomaterial can be detected using Gb3 binding reagents or reagents metabolizing Gb3 to a Gb3 metabolic product. In particular, said kits are easy-to-use kits even suitable for self-administration

In sum, the present invention opens a new avenue for diagnostics, disease monitoring and treatment control in FD using easily and repetitively available biomaterials. In this respect the present invention provides. Inter alia, for a method for detecting or diagnosing FD in a subject, which comprises detecting Gb3 deposits in easily available biomaterial obtained from said subject. Said biomaterial may be selected from the group consisting of (i) blood smear prepared from whole blood, (4i) PBMCs, and (iii) epithelial cells. In various embodiments, the amount of Gb3 positive cells detected in the chosen biomaterial is then is compared to a reference value, and when higher amount of Gb3 positive cells is detected as compared to such reference value this constitutes an indication of FD.

The terms “detecting or “diagnosing” when used herein include variations like “determining” or “identifying”. The term “detect” or detecting”, as well as the term “diagnose” or “diagnosing” when used in the context of FD refers to any method that can be used to identify subjects suffering from FD, wherein the method is based on detecting Gb3 deposits in biomaterial obtained from said subject, wherein the biomaterial is selected from the group consisting of (i) blood smear prepared from whole blood, (ii) PBMCs, and (iii) epithelial cells.

When using the term “detecting” in combination with “Gb3 deposits” in biomaterial obtained from a subject, “detect” or “detecting” is understood to refer to the amount of Gb3 deposit positive cells. The terms “amount” or “level” as used in this respect refers to a quantitative level of Gb3 deposit positive cells. The term “detection” when used herein in combination with Gb3 deposits includes both, direct detection of the target, i.e. wherein the target is detected by a signal deriving from the target) and indirect detection of the target, i.e. wherein the target is detected by a signal that does not directly derive from the target, e.g. by a signal that derives from another molecule attached to the target. The term “detection” may thus refer to the determination of the presence, subcellular localization, or amount of a given molecule or structure, such as the Gb3 deposits in the biomaterial of the present invention. The Gb3 deposits to be detected, located and/or quantified can be detected at its intracellular location in the cell obtained from a subject, for example in in the cells lysosomes, cytoplasm, membranes or another cell compartment. Accordingly, any suitable, easily applicable and reliable technique available and known to those skilled in the art that can be used to detect Gb3 deposits in the respective biomaterial described herein, thereby allowing the detection or diagnosis of FD, is comprised by the present invention. Preferably, said method allows for optical visualization or chemoelectric detection of Gb3 deposits as described elsewhere herein.

The term “subject” as used herein in the method of detecting or diagnosing FD, also addressed as an individual, refers to a living mammalian organism. Preferably, the term “subject” as used herein refers to a human subject. In some embodiments the human subject is of under 18 years of age. In fact, the methods disclosed herein are indeed particularly valuable in children, in whom predictive genetic analysis is restricted and invasive organ biopsies are often refused. According to some embodiments the subject from which the biomaterial is obtained is a patient not yet diagnosed to suffer from FD but showing first hallmarks of FD such as acral burning pain, that is triggered by heat, fever or inflammation, cardiomyopathy and nephropathy of unknown origin, repetitive cerebral stroke, particularly at young age, and gastrointestinal pain. “Of unknown origin” means in this respect that the reason of said cardiomyopathy and nephropathy cannot clearly be explained medically.

Said subject will then be examined based on the method of the present invention, i.e. by detecting the amount of Gb3 deposit positive cells in easily obtainable biomaterial from said subject, wherein the biomaterial is selected from (i) blood smear prepared from whole blood, (ii) PBMCs, and (iii) epithelial cells, in particular buccal epithelial cells. In this respect, the diagnosis or detection of FD comprises comparing the amount of Gb3 deposit positive cells detected in said biomaterial obtained from said subject to a control, wherein an increased amount of Gb3 deposits positive cells in said biomaterial as compared to a control is indicative for FD. The term “compared or comparing to a control” as used in the context of the method for detecting or diagnosing FD means that said sample can be compared to a single control sample or a plurality of control samples, such as a sample from a control subject. In any suitable manner. The term “control” as used herein can be equally substituted by the term “reference”. Said reference or control sample is preferably a sample of a subject suspected to or known to not suffer from FD. Accordingly, the control is preferably a sample from a “healthy” subject. Preferably, the control or reference measurement will be carried out in the same type of biomaterial as obtained from the subject to be diagnosed. However, since the herein described easily obtainable biomaterials all comprise Gb3 deposits in case the subject suffers from FD, i.e. all of said biomaterials are equally suitable to detect or diagnose FD, also a negative control from subjects suspected to or known to not suffer from FD can be obtained from all of said easily obtainable biomaterials. Accordingly, the control or reference sample can also be of another type of easily obtainable biomaterial as the biomaterial from the subject to be diagnosed. For example, the biomaterial from the subject to be diagnosed can be blood smear prepared from whole blood, while the biomaterial from the control subject can be a PBMC sample. The deciding factor for diagnosing or detecting FD is that the amount of Gb3 deposit positive cells is the biomaterial from the subject to be diagnosed is increased as compared to the control.

The term “increase” or “increased” as used in the context of the method for detecting or diagnosing FD described herein, in particular for the amount of Gb3 deposit positive cells, means that the respective amount or value is significantly increased as compared to the control. “Significantly increased” in this respect means that the amount of Gb3 deposit positive cells is increased by at least 5%, preferably by at least 10%, more preferred by at least 20%, even more preferred by at least 30%, even more preferred by at least 40%, even more by at least 50%, even more preferably by at least 60%, even more preferably by at least 70%, even more preferably by at least 80%, even more preferably by at least 90%, most preferably by 100% as compared to a control described elsewhere herein.

The term “biomaterial” as used in the context of the present invention refers to any cells that can be easily and repetitively obtained from a subject. According, the “biomaterial” of the present invention is preferably an “easily obtainable biomaterial”. The term “easily obtainable biomaterial” can interchangeably be used with the terms “easily accessible biomaterial” or “easily available biomaterial”. “Easily obtainable biomaterial” means in this respect that said biomaterial can be taken or achieved from a subject without the use of risky invasive methodologies or interventions, such as biopsies, in particular skin punch biopsies or organ biopsies. Hence, such “easily obtainable biomaterial” can be quickly derived from a subject or patient, “Obtained or obtainable” means in this respect that the biomaterial is derived from said subject using any methods or means known to the person skilled in the art that allow to take a sample from said subject. Preferably, for obtaining whole blood from a subject, tools like syringes or lancets are applied. Epithelial cell, in particular buccal epithelial cell, are preferably obtained using swabs like cellulose swabs. Bladder epithelial cells are preferably epithelial cells physiologically exfoliated from the bladder mucosa, which can then be derived by extracting said cells from urine.

Preferably, said “easily obtainable biomaterial” according to the present invention comprises nucleated cells that have lysosomes, such as blood cells or epithelial cells. Particularly preferred in this respect are (i) whole blood cells which can be used to prepare blood smears (ii) PBMCs and (ii) epithelial cells, such as buccal epithelial cells or bladder epithelial cells. As used within the context of the present invention, the term “whole blood” generally refers to blood from standard blood donation from which none of the elements has been removed. Accordingly, whole blood contains all the originally present in vivo constituents and may include anti-coagulants and other adjuvants. In particular, whole blood comprises red blood cells, white blood cells, plasma and platelets. In preferred embodiments whole blood according to the invention is venous peripheral blood or capillary blood. The term “venous peripheral blood” as used herein can be equivalently substituted by “whole venous blood”. “whole venous peripheral blood” or “peripheral blood” and refers to the blood pool circulating throughout the body and not sequestered within the lymphatic system, spleen, liver, or bone marrow.

According to the present invention, whole blood can be used for the purification of PMBCs. A “peripheral blood mononuclear cell” (PBMC) as described herein is any peripheral blood cell having a round nucleus. These cells consist of lymphocytes (T cells, B cells, natural idler cells) and monocytes, as opposed to erythrocytes and platelets that have no nuclei, and granulocytes which have multi-lobed nuclei. According to the present invention, said PBMCs are preferably derived from venous peripheral blood, which can be collected in 8×9 ml ethylene diamine tetra-acetic acid (EDTA) containing monovettes, which are part of the basic equipment found in medical practices and hospitals. In some embodiments said PBMCs are contained in capillary blood. The skilled person is aware of means and methods to prepare PBMCS from whole blood, such as venous peripheral blood or capillary blood obtained from a subject. From these blood samples, the PBMCs are then isolated used for detecting Gb3 deposits. To obtain a sufficient number of PBMC, withdrawal of several milliliters of blood is necessary.

In this respect the inventors of the present invention surprisingly discovered that patients with genetically approved FD exhibit huge amounts of Gb3 deposits in PBMCs when compared to a healthy control (see FIG. 1). Further, FIG. 2 illustrates that the mean percentage of Gb3 positive PBMCs is higher in men and women with FD compared to healthy controls. In addition, FIG. 3 refers to the percentage of globotriaosylceramide (Gb3) in PBMCs of men and women with Fabry disease (FD) carrying either classical (CL) or non-classical (NCL) mutations FIG. 10 shows that Gb3 deposits in PBMCs are of diagnostic value for men and women with FD carrying classical FD mutations.

However, the methods of the present invention further refer to the use of blood smear prepared from whole blood as “easily obtainable biomaterial”, which can be used to detect Gb3 deposits. In particular, the invention envisages the use of venous peripheral blood or capillary blood for the preparation of blood smears that can be used for the visualization of Gb3 deposits (see FIG. 11 and FIG. 12). The invention hence further provides for a method wherein few drops of capillary blood are obtained by, for example, a sharp lancet (similar to a portable blood sugar tests) and can be used for preparing a blood smear which allows detection of Gb3 deposits in said biomaterial. To facilitate the procedure of Gb3 detection in blood samples, the inventors investigated if blood smears immunoreacted with antibodies against Gb3 would also allow the detection of Gb3 positive blood cells. The qualitative assessment of blood samples obtained from two FD patients and two healthy controls as 10 μl whole venous blood (FIG. 11) few drops of finger stick capillary blood (FIG. 12) revealed that Gb3 deposits were also unequivocally visible in blood smear preparations. Moreover, the results may be improved when using Gb3 specific staining with e.g. its known natural ligand shiga toxin (Gallegos K M. et al. 2012 Plos One 7(2):e30388) instead of the commercial Gb3 antibodies.

The term “capillary blood” as used herein can be equivalently replaced by the term “peripheral capillary blood” and refers to peripheral blood circulating in capillaries, in particular blood capillaries. “Blood capillaries” as used herein dare the smallest blood vessels in the body, they are part of the peripheral vascular system and are from 5 to 10 micrometres (μm) in diameter, with a wall one endothelial cell thick. They convey blood between the arterioles and venues. The term “finger stick capillary blood” as herein used refers to capillary blood obtained from a subject using any tool useful to draw capillary blood by skin puncture commonly known in the art, like for example a finger stick (or finger prick) device comprising a lancet.

According to the present invention, blood smears are prepared from whole blood obtained from a subject. In other embodiments a smear can be prepared from epithelial cells, preferably buccal epithelial cells or PBMCs. Said buccal epithelial cells can be obtained using a buccal swab. PMBCs can be derived as described elsewhere herein. Hence, according to the present invention whole blood. PBMCs or epithelial cell are preferably used to prepare a smear from said cells obtained from a subject to be diagnosed. The terms “smear” or “smearing”, also sometimes named “streak” or “streaked”, as used herein refers to a sample of tissue or other material taken from part of the body of a subject which is spread thinly on a solid support for further examination, typically for medical diagnosis. According to the present invention, biomaterial like whole blood, PBMCs or epithelial cells, preferably buccal epithelial cells can be used to create a smear upon solid supports described elsewhere herein thereby allowing detection of Gb3 deposits in said smear. As the inventors show herein for the first time, a simple blood smear obtained from whole venous blood (FIG. 11) or from a drop of finger stick capillary blood (FIG. 12) allows to unequivocally detect Gb3 deposits in said thinly spread biomaterial.

Another (easily obtainable) biomaterial applicable for the means and methods described herein are epithelial cells which can be used for detecting Gb3 deposits. Preferably, said epithelial cells are buccal epithelial cells or bladder epithelial cols, wherein the bladder epithelial cells are preferably present in a urine sample. As shown by the present inventors, buccal epithelial cells, in particular buccal smear prepared using a buccal swab of a patient with Fabry disease immunoreacted with antibodies against Gb3, and Gb3 deposition could be proven in said cells (see FIG. 14). Buccal epithelial cells described herein refer to epithelial cells collected from the mouth or check of a subject. Said collected Buccal epithelial cells can be used to create a “buccal swab” or “buccal smear” on a solid support of the invention, A “buccal swab” as described herein refers to non-invasive ways to collect cells from the inside of a person's cheek. “Buccal” as used herein generally means cheek or mouth.

However, in addition to a method for detecting or diagnosing FD as described elsewhere herein, the inventors of the present invention also surprisingly discovered that the easily obtainable biomaterial of the present invention can be used for follow-up or treatment monitoring FD therapies. In particular, it could be observed that the number of Gb3 positive PBMCs of men and women with FD is highest in untreated patients, wherein the percentage of Gb3 positive PBMCs decreased under FD therapy, in particular ERT, see FIG. 4. Further, a negative correlation was found between duration of ERT and the mean percentage of globotriaosylceramide (Gb3) positive PBMC, wherein the mean percentage of Gb3 positive PBMCs was low at both visits in all patients receiving ERT and dropped in those who started ERT before visit 2 (see FIG. 5).

Accordingly, the present invention further relates to a method for treatment-monitoring or follow-up of FD in a patient, comprising comparing the amount of Gb3 deposits positive cells detected in biomaterial obtained from said patient to the amount of Gb3 deposits positive cells detected in biomaterial obtained from said patient at an earlier date, wherein said biomaterial is one of (i) blood smear prepared from whole blood. (ii) PBMCs, and (iii) epithelial cos.

The “patient” according to the method for treatment monitoring of FD is a subject, preferably a living human subject that receives any treatment for PD. Said patient may be at the start of said treatment or is already under said treatment. In some embodiments the patient has been under a certain treatment for a while which did not lead to any improvement of the disease or only slightly improved the disease. Hence, the “patient” under treatment monitoring can be a patient restarting treatment.

In some embodiments the chosen treatment is an ERT comprising agalsidase. Specific treatment known to those skilled in the art consists of ERT with recombinant αGAL-A (agalsidase). Two agalsidase products are currently available on the market, agalsidase alfa and agalsidase beta. Agalsidase alfa is manufactured by Shire Human Genetic Therapies (Cambridge, Mass., USA, now Takeda) from human cell lines and administered every two weeks by intravenous infusion (over 40 min) at a dose of 0.2 mg/kg. Agalsidase beta is manufactured by Genzyme Corporation, which was recently acquired by Sanofi-Aventis (Paris. France), from CHO (Chinese hamster ovary) cells and administered every two weeks by intravenous infusion at a dose of 1 mg/kg at an initial rate of 0.25 mg/min. Both products have been approved for use in the European Union since 2003, but only agalsidase beta has been cleared by the U.S. Food and Drug Administration (FDA) for use in the USA (Alegra et al. 2012 Genet Mol 35(4 Suppl): 947-954.) Biol.

The term “treat”, “treating”, or “treatment” as used herein means to reduce, stabilize, or inhibit the progression of the symptoms associated with FD. Said symptoms may include episodes of pain, especially in the hands and feet, clusters of small, dark red spots on the skin called angiokeratomas, a decreased ability to sweat (hypo- to anhidrosis), cloudiness of the cornea of the eye (cornea verticillata), and hearing loss, internal organs, such as the kidneys, heart or brain, may also be affected, leading to progressive renal impairment, cardiomyopathy, and cerebral strokes. Milder forms of FD may appear later in life and affect only the heart or kidneys (Mehta A & Hughes D A. Fabry Disease. GeneReviews. 2017). Those patients in need of treatment include those already with the disorder as well as those prone to having the disorder. Preferably, a treatment reduces, stabilizes, or inhibits progression of a symptom that is associated with the presence and/or progression of a disease or pathological condition. “Treat”, “treating”, or “treatment” refers to both therapeutic treatment and prophylactic or preventative measures, wherein the object is to prevent, slow down (lessen) or at least partially alleviate or abrogate an abnormal, including pathologic, condition in the organism. Those in need of treatment include those already with the disorder as well as those prone to having the disorder or those in whom the disorder is to be prevented (prophylaxis).

In sum, the treatment which is monitored according to the method of treatment monitoring of the invention comprises a compound reducing Gb3 deposits in Gb3 deposit positive cells or a compound reducing the production of Gb3 deposits. The term “reducing Gb3 deposits in Gb3 deposit positive cells” as used herein in this respect refers to an apparent, i.e. a significant reduction of Gb3 deposits in Gb3 deposit positive cells when compared to the amount of Gb3 deposit positive cells detected in biomaterial obtained from said subject at an earlier date. ‘Significantly’ means that the Gb3 deposits in Gb3 deposit positive cells are reduced by at least 10%, more preferred by at least 20%, even more preferred by at least 30%, more preferred by at least 40%, more preferred by at least 50%, more preferred by at least 60%, more preferred by at least 70%, more preferred by at least 80%, more preferred by at least 90%, most preferred by 100% when compared to the amount of Gb3 deposit positive cells detected in biomaterial obtained from said subject at the earlier date.

The term “treatment monitoring” as used herein can be interchangeably used with the term “follow-up” and refers to the act of detecting the amount of Gb3 deposits in the herein described biomaterials at intervals during therapy, thereby using the means and methods according to the present invention. Said intervals generally comprise days, weeks (i.e. short term follow-up), months and even years (i.e. long-term follow-up). Preferably, the treatment is monitored every second day within the first week, more preferably daily within the first week of treatment. The “treatment monitoring” thereby aims at regulating a FD treatment, changing an ongoing FD treatment with a more appropriate one and/or controlling the response to the ongoing FD treatment.

However, apart from ERT comprising alpha-galactosidase, the prior art also refers to other promising therapies to treat FD, such as chaperone therapy or substrate reduction. Thus, accordingly to the method of treatment monitoring said treatment any one of an enzyme. A “chaperone therapy” as mentioned herein refers to a therapy comprising pharmacological chaperones to facilitate the proper folding of the mutant alpha-Gal enzyme by binding to its active site, thereby improving its stability and trafficking to the lysosomes. A chaperon therapy as used according to the invention can include the pharmacological chaperone migalastat.

“Substrate reduction therapy” as referred to herein is a therapy that reduces the amounts of the substrate of a certain enzyme. In the case of FD, the activity of the glucosylceramide synthase GCS enzyme is inhibited, thereby blocking the formation of glucosylceramide (GL-1) which then prevents the production of Gb3, the substrate of alpha-galactosidase A. In this way, the therapy ensures that lack of this enzyme in FD is no longer a problem. One compound to be used for “substrate reduction therapy” is lucerastat that has been reported in a phase 1 clinical study (NCT02930655) to significantly decrease the amounts of three substrates: GL-1, lactosylceramide, and Gb3. Hence, in some embodiments, the treatment may comprise the use of a therapy which is still in the clinical trial phase, such as a substrate reduction treatment comprising lucerastat currently under investigation. In some embodiment of the method for treatment monitoring, said treatment comprises agalsidase, migalastat, or lucerastat, or a combination thereof.

Another treatment according to the method of treatment monitoring of FD comprises gene therapy. “Gene therapy” as used herein refers to any therapeutic delivery of nucleic acid into a patient's cell as a drug, thereby treating FD. In particular, gene therapies aim at altering a disease-causing gene or introducing a healthy copy of a mutated gene to the body. Hence, according to the present invention, gene therapy aims at altering mutations in the α-GAL gene leading to FD phenotype as described elsewhere herein, or at introducing a healthy copy of the α-GAL gene.

Patients suffering from FD carry a mutation in the α-GAL gene, leading to a reduce amount or no production of alpha-galactosidase A which normally metabolizes Gb3. Hence said mutations lead to accumulation of Gb3 and the production of Gb3 deposits in several somatic cells. Further, the examples of the present invention underline that the provided methods for detecting or diagnosing FD and treatment monitoring FD are particularly useful in subjects or patient carrying classical mutation. Hence, according to the method for treatment monitoring and detection/diagnosis of FD, the subject or patient preferably carries a mutation in the α-GAL gen leading to a FD phenotype. Preferably, said phenotype is a classical FD phenotype, FIG. 15 illustrates the genetic distribution in the study population

According to another aspect, the present invention also refers to a method of prognosis of FD in a subject, comprising detecting Gb3 deposits in biomaterial obtained from said subject, wherein said biomaterial is selected from the group consisting of (i) blood smear prepared from whole blood, (ii) PBMCs, and (ii) epithelial cells. In this respect, the prognosis comprises detection of the initial Gb3 load, which can be used to predict the progress of the disease. The “Gb3 load” as used herein is the amount of Gb3 deposit positive cells detected in the biomaterial of the invention which is obtained from said subject. “Initial” means that the load is detected when making the first analysis for a subject. Hence, a low initial Gb3 load is indicative for a milder progress of FD, while a higher initial Gb3 load is indicative for a more severe progress of FD. A “mild progress” means in this respect that the subject will develop less symptoms of FD (characterizing the FD phenotype), while a “severe progress” means that the subject will develop more symptoms of FD (characterizing the FD phenotype). Symptoms of FD are defined elsewhere herein.

In this respect, the prognosis of FD comprises comparing the amount of Gb3 deposit positive cells detected in biomaterial obtained from a subject to a control. Hence, a “low Gb3 load” or a high “Gb3 load” means that the amount of Gb3 deposit positive cells is increased when compared to said control. The term “compared or comparing to a control” as used in the context of the method for the prognosis of FD means that said sample can be compared to a single control sample or a plurality of control samples, such as a sample from a control subject, in any suitable manner. The term “control” as used herein can be equally substituted by the term “reference”. Said reference or control sample is preferably a sample of a subject suspected to or known to not suffer from FD. Accordingly, the control is preferably a sample from a “healthy” subject. Preferably, the control or reference measurement will be carried out in the same type of biomaterial as obtained from the subject for whom a prognosis should be made. However, since the herein described easily obtainable biomaterials all comprise Gb3 deposits in case the subject suffers from FD, i.e. all of said biomaterials are equally suitable to detect or diagnose FD, also a negative control from subjects suspected to or known to not suffer from FD can be obtained from an of said easily obtainable biomaterials. Accordingly, the control or reference sample can also be of another type of easy obtainable biomaterial as the biomaterial from the subject to be diagnosed. For example, the biomaterial from the subject to be diagnosed can be blood smear prepared from whole blood, while the biomaterial from the control subject can be a PBMC sample. The deciding factor for diagnosing or detecting FD is that the amount of Gb3 deposit positive cells is the biomaterial from the subject to be diagnosed is Increased as compared to the control.

The term “subject” as used herein in the method for the prognosis of FD, also addressed as an individual, refers to a living mammalian organism. Preferably, the term “subject” as used herein refers to a human subject. In some embodiments the human subject is of under 18 years of age. According to some embodiments, the subject from which the biomaterial is obtained is a patient not yet diagnosed to suffer from FD but showing first hallmarks of FD as defined elsewhere herein. Detecting the initial Gb3 load in biomaterial obtained from said subject will help to get a prognosis on the onset or on the further progress of FD. This method will also help to get a prognosis on the progress of FD under a respective therapy applied which is described elsewhere herein. The lower the initial Gb3 load, the more promising or successful will be the therapy applied to said subject.

The term “mutation leading to a (classical) FD phenotype” as used herein can be replaced by the term “classical mutation” and indicates a mutation which is known to be associated with typical symptoms and signs of FD such as early onset and multi organ disorder. Contrary thereto, the term “non-classical mutation” as opposed to “classical mutation” used herein can be replaced by the term “mutation leading to a non-classical FD phenotype” and indicates a mutation associated with late onset of FD or with the affection of predominantly one organ (van der Tol at al. 2017 JDM Rep 17:83 90). In some preferred embodiments a mutation leading to a classic FD phenotype is a nonsense mutation. The term “nonsense mutation” as used herein refers to a mutation in which a sense codon that corresponds to one of the twenty amino acids specified by the genetic code is changed to a chain-terminating codon. Hence, “nonsense mutations” lead to no production of alpha-galactosidase. However, also patients carrying “missense mutations” can develop severe symptoms of FD. A “missense mutation” is a mutation leading to the production of alpha-galactosidase having reduced function. Hence, according to the present invention, the mutation leading to F phenotype is a nonsense mutation or a missense mutation, preferably a nonsense mutation. The mutation in the α-GAL gene is preferably any mutation associated to Fabry Disease that can be found on the portal https://lih16.u.hpc.mssm.edu/pipeline/is/dbFabry/Mutation.html#.

According to the method of treatment monitoring, a decreased amount of Gb3 deposit positive cells as compared to the amount of Gb3 deposit positive cells detected an earlier date indicates a positive treatment effect, while no change or an increased number of Gb3 deposit positive cells as compared to the amount of Gb3 deposit positive cells detected at an earlier date indicates no treatment effect. The term “increase” or “increased” as used in the context of the method for treatment monitoring of FD described herein, in particular for the amount of Gb3 deposit positive cells, means that the respective amount or value is significantly increased as compared to the amount of Gb3 deposit positive cells detected at an earlier date. “Significantly increased” in this respect means that the amount of Gb3 deposit positive cells is increased by at least 5%, preferably by at least 10%, more preferred by at least 20%, more preferred by at least 30%, more preferred by at least 40%, by at least 50%, even more preferably 30%, even more preferably 40%, even more preferably 50%, even more preferably 60%, even more preferably 70%, even more preferably 80%, even more preferably 90%, most preferably by 100% as compared to the amount of Gb3 deposit positive cells detected at an earlier date. The term “decrease” or “decreased” as used in the context of the method for treatment monitoring of FD described herein, in particular for the amount of Gb3 deposit positive cells, means that the respective amount or value is significantly decreased as compared to the amount of Gb3 deposit positive cells detected at an earlier date. “Significantly decreased” in this respect means that the amount of Gb3 deposit positive cells is decreased by at least 5%, preferably by at least 10%, more preferred by at least 20%, more preferred by at least 30%, more preferred by at least 40%, more preferred by at least 50%, more preferred by at least 60%, more preferred by at least 70%, more preferred by at least 80%, more preferred by at least 90%, most preferred by 100% as compared to the amount of Gb3 deposit positive cells detected at an earlier date. “No change” means that the amount of Gb3 deposit positive cells is the same as compared to the amount of Gb3 deposit positive cells detected at an earlier date. In preferred embodiments, the method for treatment monitoring comprises detecting Gb3 deposit positive cells in said biomaterial.

So far, the detection of Gb3 deposits other than in organ biopsies plays a subordinate role in the prior art in the diagnosis and follow-up of FD. However the detection of Gb3 deposits in easily available biomaterial such as blood cells brings several advantages. The methods described herein, such as a simple blood test allowing the direct detection of Gb3 accumulations may revolutionize FD diagnostics. Compared to the currently available diagnostic tools, the herein described methods give fast results and are inexpensive to carry out. The visualization of Gb3 deposits in PBMCs, blood smears or buccal epithelial cells of patients with FD revealed several plausible results that underline the robustness, reliability, and validity of the method (see Examples 1 and 2). These results span the spectrum from highest cellular Gb3 deposits in untreated men carrying classical nonsense mutations with low α-GAL activity and high serum lyso-Gb3 levels to only single Gb3 carrying cells found in healthy controls (for a correlation between Gb3 positive PBMC and α-GAL activity and between Gb3 positive PBMC and lyso-Gb3 (see FIG. 6 and FIG. 7). These findings underline the diagnostic and prognostic potential of blood Gb3 assessment in FD diagnosing and/or treatment monitoring. Gb3 deposits according to the present invention comprise the acylated form of Gb3. However, in some embodiments also the deacylated form of Gb3, called “lyso-Gb3” may be detected in the biomaterial of the present invention in addition to Gb3. High levels of Lyso-Gb3 generally correlate with high FD activity, although its diagnostic significance is still under investigation (see also FIG. 8)

The assessment of the mean percentage of Gb3 positive biomaterial such as PBMC blood cells also opens a new window for follow-up investigations and monitoring of FD patients. By repetitive assessment of easily available biomaterial such as blood cells it will become possible to non-invasively monitor disease development and treatment response. Currently, the effect of intravenous or oral FD specific therapies cannot be directly assessed, not by measuring α-GAL activity and not by monitoring lyso-Gb3 (FIG. 9), but is indirectly assumed looking at the results of organ diagnostics. Particularly during ERT, treatment response can peter out e.g. due to the development of antibodies in the course of the treatment (Wilcox et al., 2012), which would require switching to an alternative ERT or to oral treatment.

Gb3 assessment in easily available biomaterial such as blood smears prepared from whole blood. PBMCs or epithelial cells may provide the basis for a bedside diagnostic test, i.e. a test for self-administration described elsewhere herein. This is particularly valuable in children, where predictive genetic analysis is restricted and invasive organ biopsies are often refused. Furthermore, the detection of Gb3 deposits in blood cells can be of help when dealing with otherwise equivocal results from molecular and genetic analysis.

The investigation of Gb3 deposits in easily available biomaterial is complementary to the investigation of α-GAL activity allowing to assess its functional consequences with an easy-to-apply method. Seeing that low enzyme activity is also associated with a relevant deposition of Gb3 in easily available biomaterial such as PBMC blood cells may help making a decision on starting an FD specific treatment particularly in women who mostly present with a later-onset and milder clinical phenotype. Furthermore, the detection of Gb3 deposits in blood cells like PBMCs, blood smears or epithelial cells, can be of immense help when dealing with otherwise equivocal results from the molecular and genetic analysis.

Determining Gb3 load in easily available biomaterial like blood smears from whole blood, PBMCs, or epithelial cells also has the immense potential to be included as an objective and easy-to-apply outcome measure in pharmaceutical studies. Monitoring changes in Gb3 load under treatment is not possible so far, but may become a very useful tool to be included in future studies. Such an outcome parameter will also help to improve data quality.

The methods of the present invention preferably comprise (a) depositing the aforementioned biomaterial obtained from a subject or patient on a solid support thereby immobilizing said biomaterial, and (b) detecting Gb3 deposit positive cells in said biomaterial. The term “depositing has been described elsewhere herein and refers to the act of placing the biomaterial on a solid support onto which the biomaterial can be analyzed. The term “immobilizing” refers to the fixation of said biomaterial on the solid support. Said immobilizing step preferably comprises a fixing agent. “Fixing agents” as used herein are chemical agents used to preserve structures in a state (both chemically and structurally) as close to living tissue as possible by terminating any ongoing biochemical reactions and increasing the sample's mechanical strength or stability. Fixative agents as used herein include but are not limited to: aldehydes such as formaldehyde or glutaraldehyde, alcohols like ethanol, methanol, acetone or acetic acid.

The methods for detecting or diagnosing FD or treatment monitoring or prognosis of FD as well as the kit described elsewhere herein comprise the step of detection of Gb3 deposits or Gb3 deposit positive cells in applied biomaterial. The term “optical visualization” or “optic visualization” as used herein is a visualization technique involving an optical system. The term “optical system” or “optic system”, as herein used is a system suitable to be used for imaging, for example imaging of a labeled antibody, a chromatic substrate or chromatic metabolic product. The term “optical” or “optic”, as used herein, preferably refers to visible light but is generally not limited to it. The term may also refer to infrared, ultraviolet and other regions of the electromagnetic spectrum. The term “chemoelectric detection” or “electrochemical current detection” can be used interchangeably, and they refer to detection of an electrochemical response in the form of a decaying electrical current which comes from a biochemical reaction as described for example in US20060040333A1. Such a “chemoelectric detection” envisages that the biomaterial (for example selected from a blood smear prepared from whole blood, PBMCs, or epithelial cells) reacts with and alpha-galactosidase enzyme, this reaction in turn produces an electrical response in the form of a decaying electrical current, which is then converted by electronics into a digital signal that is processed to determine the analyte test value that corresponds to the signal.

The methods and kits of the present invention further comprise contacting the immobilized biomaterial with a Gb3 binding agent. A “Gb3 binding agent” as used herein is any agent direct reacting with Gb3. Hence, Gb3 binding agents can react with Gb3 deposits in Gb3 deposit positive cells. Preferably, said reagents are Gb3 specific antibodies or Gb3 natural ligands. For the immunoreaction to Gb3 comprising Gb3 specific antibodies different possibilities known in the art can be used. For example a primary antibody specific for Gb3 and conjugated with a detectable label allowing for direct visualization can be used or, in alternative, a primary Gb3 antibody followed by secondary antibody conjugated with a detectable label can be used. The immunoreacted cells can then be visualized using any visualization technique, like light microscopy and the data obtained can then be analysed using a software of choice, known to those skilled in the art.

As described herein, the step of contacting the biomaterial with a Gb3 binding agent is preferably carried out at conditions that allow specific interaction of the Gb3 specific antibody and target it specifically binds to. Such conditions are well known to the person of skill in the art. Washing steps typically follow the contacting step of an antibody to its antigen, and the skilled person knows how and when to apply said washing steps. Said (primary) antibody will specifically interact with the Gb3. Preferably, Gb3 specific antibodies comprise a label. Hence, also primary antibodies, might be conjugated to a detectable label. In such case the interaction can be detected, monitored and quantified by measuring or observing the reporter signal obtained from the detectable label. Preferably, said label comprises a fluorescence entity, such as a fluorescence label. For example, if the detectable label is a fluorescent moiety, fluorescence can be measured and observed upon excitation.

In the context of the present invention, the primary antibody specific to the Gb3 deposits in the biomaterial of the invention, may be specifically recognized by a (secondary) antibody, which carries a detectable label, such as a fluorescent label. The method of detection of the Gb3 deposits in the biomaterial of the invention may thus comprise the step of contacting the Gb3 containing biomaterial with a primary antibody and subsequently with a secondary antibody conjugated to a detectable label and specifically binding to the primary antibody bound to the Gb3. The immunoreacted biomaterial can then be visualized using any light microscopy instrument and the data obtain can then be analyzed using a software of choice known to those skilled in the art.

The antibody used to detect Gb3 deposits may be conjugated to a detectable label. In general, such a “detectable label” or “label” as used herein may be any appropriate chemical substance or enzyme, which directly or indirectly generates a detectable compound or signal in a chemical, physical, optical, or enzymatic reaction. For example, a fluorescent or radioactive label can be conjugated to the antibody to generate fluorescence or X-rays as detectable signal. Alkaline phosphatase, horseradish peroxidase and β-galactosidase are examples of enzyme labels (and at the same time optical labels), which catalyze the formation of chromogenic reaction products. In a preferred embodiment, the detectable label refers to detectable entities that can be used for the detection of the target of interest in microscopy, immunohistochemistry or flow cytometry. Preferably, the label does not negatively affect the characteristics of the antibody to which the label is conjugated. Examples of labels are fluorescent labels such as phycoerythrin, allophycocyanin (APC), Brilliant Violet 421, Alexa Fluor 488, coumarin or rhodamines to name only a few. There are many types of detectable labels, including a fluorescent label, a chromophore label, an isotope label, or a metal label, with a fluorescent label being preferred. The detection of Gb3 deposits via the methods and kits of the invention may be achieved by contacting the biomaterial containing or suspected to contain Gb3 deposits with an antibody conjugated to a detectable label and detecting the signal of the detectable label. For a fluorescent label, this means detection of emitted light upon excitation of the fluorescent label. Non-exhaustive examples for suitable fluorescent labels are “green” emitters (Atto488, Alexa488, Cy2, etc.). “orange” emitters (Atto542, alexa555, Cy3, etc.), “Red-far-Red” emitters (Alexa633, Atto 647N, Cy5, etc.), infrared emitters (Atto700, LiCor IRDye700, LiCor IRDye800, etc.), ultraviolet absorbing fluorescent dyes (Atto390 or Alexa405). A fluorescent label may also be a fluorescent protein, such as GFP, eGFP, YFP, RFP, CFP, BFP, mCherry, or near-infrared fluorescent proteins. Non-exhaustive examples for a suitable chromophore label are alkaline phosphatase or peroxidase exposed to TMB (3,5,5′ tetramethylbenzidine), DAB (3,3,4,4′ diaminobenzidine), and 4CN (4-chloro-1-naphthol). ASTS (2,2′-azino-di [3-ethyl-benzthiazoline] sulfonate), OPD (o-phenylenediamine), and to BCIP/NST (5-bromo-4-chloro-3-indolyl-phosphate/nitroblue tetrazolium). Non-exhaustive examples for isotope labels are 13C, 15N, 19F, 27Al, 11B, 127I or different Lanthanides isotopes. Non-exhaustive examples for a metal label are Au, Pd, Pb, Pt Ag, Hg and Os. The label may be a direct label, i.e. a label that is directly detectable. Alternatively, the label may be an indirect label. i.e. a label which is an affinity tag (or epitope tag) that can be specifically bound by another specific binding partner that is conjugated to another detectable label, such as a fluorescent or chromophore label. Examples of suitable epitope tags include, but are not limited to, FLAG-tag, Strep-tag, Myc-tag, HA-tag, 162VSV-G-tag, HSV-tag, VS-tag, SPOT-tag, BC2 tag and EPEA tag. The antigen may also be a protein, for example, glutathione-S-transferase (GST), maltose binding protein (MBP), chitin binding protein (CBP) or thioredoxin as an antigen. The detectable label may further be a nucleic acid, such as an oligonucleotide having a recognition sequence. Such a recognition sequence may be a random sequence. This random sequence may be barcode sequence that has been incorporated into the nucleic acid molecules and can be used to identify the target molecule that has been conjugated with said nucleic acid. An ‘antibody may be conjugated to a detectable label’ may also mean that the antibody itself is the detectable label. This may imply that the antibody is an affinity target that can be specifically recognized by another specific binding partner that specifically binds to the antibody. For example, such a specific binding partner may be an antibody that specifically recognizes mouse IgG. Such a specific binding partner may further be conjugated to a detectable label, such as a fluorescent label.

According to the means and methods described herein, the Gb3 binding agent may also comprises the use of aptamer-target-binding technology. When applying aptamer-target-binding technology, Gb3 may be identified by a class of small nucleic acid ligands (aptamers). In some embodiments the aptamers are composed of RNA having high specificity and affinity for their targets. In some embodiments the aptamers are composed of single-stranded DNA oligonucleotides having high specificity and affinity for their targets. Similar to antibodies, aptamers interact with their targets by recognizing a specific three-dimensional structure and are thus termed “chemical antibodies,” In contrast to protein antibodies, aptamers offer unique chemical and biological characteristics based on their oligonucleotide properties.

A “Gb3 natural ligand” as described herein as Gb3 binding agent refers to a compound or molecule of natural sources, e.g., plants, animals, bacteria, etc. not produced or engineered by humans, which is able to specifically recognize and bind Gb3. In some preferred embodiments, such Gb3 natural ligands include, but are not limited to, shiga toxin. Shiga toxins are a family of related toxins with two major groups. Stx1 and Stx2, expressed by genes considered to be part of the genome of lambdoid prophages. The most common sources for shiga toxin are the bacteria S. dysenteriae and the shigatoxigenic serotypes of Escherichia coli (STEC). The glycolipid Gb3 has been reported to be the receptor for both toxins, the B subunits pentameric portion of the shiga toxin has been shown to bind Gb3 in host cell membranes (Gallegos K M. et al 2012 Plos One 7(2):e30368). The inventors provide herein a Gb3 visualization method by using Gb3 natural ligands to improve the staining of Gb3 deposits given the lack of commercially available antibody having consistent and satisfactory staining efficiency.

As herein used herein the term “staining” refers to the use of Gb3 binding agents as defined herein for visualizing Gb3 deposits. Therefore, a “staining agent” can be a Gb3 specific antibody or a Gb3 natural ligand.

Accordingly, the present invention also refers to the use of a Gb3 specific natural ligand as described elsewhere herein for the detection of Gb3 deposits in biomaterial described elsewhere herein. Preferably, said Gb3 specific natural ligand is shiga toxin, but the invention is not limited thereto.

The methods and kits of the present invention may further comprise contacting the immobilized biomaterial with a reagent metabolizing Gb3 to a Gb3 metabolic product thereby allowing for detection of Gb3 deposit positive cells. Such “reagent metabolizing Gb3 to a Gb3 metabolic product” can be any compound converting Gb3 to its metabolite. Such reagent can be, for example, an alpha-galactosidase enzyme. The metabolic product allowing for optical visualization of the Gb3-deposits positive cells include but are not limited to chromatic metabolic products for detection with an optical system, a metabolic product for reaction with further substrate thereby allowing for optical visualization, or a metabolic product allowing for chemoelectric detection as described elsewhere herein.

The term “Gb3 deposits” as used herein refers to intracellular Gb3 present in cells obtained from a patient suffering from FD. Preferably, Gb3 deposits are present in the easy obtainable biomaterial according to the present invention. Intracellular Gb3 deposits can refer to Gb3 contained in cytoplasm, lysosomes, membranes or other cellular compartments. “Gb3-positive cell” or “Gb3-positive biomaterial” as used herein refers to biomaterial or cells wherein Gb-3 deposits are detectable by the methods and kits of the invention.

The present invention also relates to a kit for detecting or diagnosing FD or treatment monitoring or prognosis of FD. The kit may comprise components necessary to carry out the methods of the present invention. The kit can be used for self-administration by physicians dealing with adult and pediatric Fabry patients such as general practitioners, cardiologists, nephrologists, and neurologists. In this respect, Gb3-deposits contained for example in few drops of capillary blood can be visualized using a biochemical reaction that leads to a change in color of Gb3 metabolites similar to those used in blood sugar test devices or tests for pregnancy. Such chemoelectric detection is described elsewhere herein. Optical systems or optic systems for automatic Gb3 detection are also potential applications. Such optical systems are also described elsewhere herein. FIG. 13 summarizes how the test kits of the present inventions works, thereby allowing for automatization of the diagnosis and treatment monitoring of FD.

The kit of the present invention comprises (a) a first solid support for depositing biomaterial, and (b) a Gb3-binding agent or a reagent metabolizing Gb3 to Gb3 metabolic products allowing for detection of Gb3 deposits in said biomaterial.

The “solid support” used according to the present invention necessarily allows to detect Gb3 deposits in biomaterial deposited on said support, thereby using any of the detection methods described elsewhere herein. The solid support may comprise, in its entirety or in some parts, organic or inorganic polymer material allowing for deposition of biomaterial. The solid support may also comprise, in its entirety on in some parts, of a heat-resistant plastic material. According to a preferred embodiment the solid support is a glass slide. The term “solid support” as used herein preferably refers to a thin, flat piece of material for deposition of biomaterial. The glass slide for use in methods and kits of the present invention may be a microscope slide, intended as a thin flat piece of glass, used to hold objects and which allows for said object to be examined using a microscope. Typically the object is mounted (secured) on the slide, and then both (the slide and the object secured onto it) are inserted together in the microscope for viewing. In the present invention the object secured onto the glass slide is biomaterial containing or suspected to contain Gb3 deposit positive cells. The solid support according to the present invention may consist, in its entirety or in part, of hydrophobic surface. A surface is “hydrophobic” if an aqueous-medium droplet applied to the surface does not spread out substantially beyond the area size of the applied droplet. That is, the surface acts to prevent spreading of the droplet applied to the surface by hydrophobic interaction with the droplet. The surface of the solid support described herein may have or may be formed to have a relatively hydrophobic character, i.e., one that causes aqueous medium deposited on the surface to bead. A variety of known hydrophobic polymers, such as polystyrene, polypropylene, or polyethylene have desired hydrophobic properties, as do glass and a variety of lubricant or other hydrophobic films that may be applied to the support surface. The solid support may be a non-porous solid support. Said non-porous solid support comprises a plate or plates, a well or wells, a microliter well or microtiter wells, a depression or depressions, a tube or tubes, or a cuvette or cuvettes. The solid support may be a solid support that has been treated with a surface treatment agent, a blocking agent, or both. Accordingly, the term “solid support” as herein used preferably refers, but is not limited to, a glass side, a plastic slide, a plexiglass slide or any surface able to support the biomaterial in a way that this can be examined using any detection system for detecting Gb3 deposits in said biomaterial.

The term “depositing” as used within the context of the methods and kits described herein refers to the act of placing the biomaterial on the solid support described elsewhere herein, onto which the biomaterial can be analyzed with any detection method allowing detection of Gb3 deposits in said biomaterial. Depositing of the biomaterial onto the solid support might be achieved through the use of a laboratory pipet, a tool commonly used in chemistry, biology and medicine to transport a measured volume of liquid, often as a media dispenser. In some embodiments depositing of the biomaterial on solid support additionally comprises smearing the biomaterial onto the solid support.

The kit according to the present invention may also comprise a Gb3-binding agent allowing for visualization of Gb3 deposits, or a reagent metabolizing Gb3 to a Gb3 metabolic product allowing for visualization as described elsewhere herein. The kit may also comprise a solid support comprising the Gb3 binding agents and/or the Gb3 metabolizing agents of the invention immobilized or attached to the solid support. The kit herein described may also comprise an antibody, optionally conjugated to a detectable label as described elsewhere herein, preferably an optically detectable label. The kit of the present invention may optionally comprise a Gb3 natural ligand as described elsewhere herein, such as a shiga toxin, allowing for visualization of Gb3 deposits. Alternatively, the kit may comprise a Gb3 metabolizing agent as described elsewhere herein, such as alpha-galactosidase. The kit may also comprise buffers and reagents necessary for the detection methods of the present invention. Furthermore, the kit described herein may also comprise at least one (secondary) specific antibody as described elsewhere heroin.

The kit may also comprise any tools useful to obtain biomaterial from a subject. In some embodiments the kit may comprise a tool to draw venous peripheral blood or capillary blood by skin puncture from a subject. Such tool may be syringes or lancets, like for example a finger stick device. A “fingerstick device” also called “finger prick device” or ““lancing device” as used herein refers to a device comprising a lancet and used in a procedure in which the skin, for example the skin of a finger, is pricked with said lancet to obtain a small quantity of capillary blood for testing. A “lancet” as used herein refers to a double-edged blade or needle that can be used to make punctures. Lancets can be disposable. A lancing device can be used to prick the finger or in general the skin of a subject from which the biomaterial has to be obtained. The terms “fingerstick device” also called “finger prick device” or lancing device” as defined herein may be used interchangeably. In some embodiments the kit may comprise a tool to obtain buccal epithelial cells from a subject. Such tool may be a swab. A “swab” as used herein refers to a small piece of soft, absorbent material, such as gauze, or cellulose, used to clean wounds, apply medicine, or take samples from a subject. Such swabs can be “buccal swabs” used to obtain buccal epithelial cells from a subject. Such buccal swabs may be attached to a stick or wire to aid access.

The kit may also comprise a “dyeing agent”. Dyeing agents as commonly used in the art are agents used to highlight structures in biological tissues for viewing, often with the aid of different optical systems or microscopes. Dying agents may be used to define and examine bulk tissues, cell populations (classifying different blood cells, for instance), or organelles within individual cells. In biochemistry it involves adding a class-specific (DNA, proteins, lipids, carbohydrates) dye to a substrate to qualify or quantify the presence of a specific compound.

Another aim of the present invention is the generation of an analogous test kit system preferably for self-administration (named FABRYSWAB), wherein preferably buccal epithelial cells are used for Gb3 detection. This kit system works in analogy to the blood smear based kit FABRYSTIX described in FIG. 13, but comprises usage of swabs to deposit and distribute epithelial cells on the sold support comprised by said kit. FIG. 14 shows the very promising result of Gb3 visualization in buccal epithelial cells, which confirm suitability of buccal epithelial cells in such a kit.

The biomaterial according to the methods and kits of the present invention is preferably permeabilized or lysed. Lysing or permeabilizing agents used according to the invention aim at partial (permeabilizing) or complete (lysing) destruction of the integrity of the cell membrane, thereby allowing for a better detection of Gb3 deposits in said biomaterial. Lysing or permeabilizing agents include but are not limited to commonly used agents, such as: organic solvents, methanol and acetone, and detergents such as saponin. Triton X-100 and Tween-20. As used herein, permeabilizing agents are agents allowing antibodies and other Gb3 binding agents to pass through the cellular membrane and enter the cell.

Unless otherwise stated, the following terms used in this document, including the description and claims, have the definitions given below.

Those skilled in the art will recognize, or be able to ascertain, using not more than routine experimentation, many equivalents to the specific embodiments of the invention described herein. Such equivalents are intended to be encompassed by the present invention.

It is to be noted that as used herein, the singular forms “a”, “an”, and “the” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or ‘approximately’ as used herein means within 20%, preferably within 10%, and more preferably within 5% of a given value or range. It includes, however, also the concrete number. e.g., about 20 includes 20.

Throughout this specification and the claims which follow, unless the context requires otherwise, the word “comprise”, and variations such as “comprises” and ‘comprising’, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not material affect the basic and novel characteristics of the claim.

In each instance herein any of the terms “comprising”, “consisting essentially of and consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### BRIEF DESCRIPTION OF THE DRAWINGS

FIG. 1: A) Nuclear stain (blue) of peripheral blood mononuclear cells (PBMC) of an untreated patient with genetically approved Fabry disease (FD). B) Immunoreaction with an antibody against globotriaosylceramide (Gb3) reveals Gb3 deposits (green) in several PBMC. C) Merged image of A) and B). D) Nuclear stain (blue) of PBMC of a healthy control. E) Immunoreaction with an antibody against Gb3 reveals one cell with Gb3 deposits (green). F) Merged image of D) and E). Yellow arrows indicate Gb3 positive PBMC. Scale bar: 50 μm.

FIG. 2: Bar graphs illustrate the mean percentage of globotriaosylceramide (Gb3) positive peripheral blood mononuclear cells (PBMC) of men (M) and women (F) with Fabry disease (FD). The mean percentage of Gb3 positive PBMC is higher in men and women with FD compared to healthy controls (Co). ***p<0.001, **p<0.01.

FIG. 3: Bar graphs illustrate the mean percentage of globotriaosylceramide (Gb3) positive peripheral blood mononuclear cells (PBMC) of men (M) and women (F) with Fabry disease (FD) carrying either classical (CL) or non-classical (NCL) mutations. The mean percentage of Gb3 positive PBMC is higher in men and women with classical FD associated mutations compared to healthy controls (Co). ***p<0.001, **p<0.01.

FIG. 4: Bar graphs illustrate the mean percentage of globotriaosylceramide (Gb3) positive peripheral blood mononuclear cells (PBMC) of men (M) and women (F) with Fabry disease (FD) carrying classical (CL) mutations and with or without enzyme replacement therapy (ERT). In men, the mean percentage of Gb3 positive PBMC decreased with ERT: the number was highest in untreated men (p<0.001), less in those with ERT>8 days before (p<0.01), and lowest in men with ERT up to eight days before blood withdrawal compared to healthy controls (Co). In women, the mean percentage of Gb3 positive PBMC was also highest without ERT compared to healthy female controls (Co). ***p<0.001, **p<0.01, **p<0.05.

FIG. 5: A) A negative correlation was found between duration of enzyme replacement therapy (ERT) and the mean percentage of globotriaosylceramide (Gb3) positive peripheral blood mononuclear cells (PBMC) in men with Fabry disease (FD) carrying classical mutations (Spearman correlation coefficient −0.457, p<0.05). B) Of n=15 male (M) and female (F) patients, a second blood sample was obtained at a follow-up visit (visit 1, visit 2). These patients carried classical (CL) and non-classical (NCL) mutations and were either untreated (no ERT) or received ERT. Mean percentage of Gb3 positive PBMC was low at both visits in all patients receiving ERT and dropped in those who started ERT before visit 2. In contrast mean percentage of Gb3 positive PBMC remained high in a male (#6) and female (#15) patients carrying CL mutations without ERT. *ERT was started after visit 1. **ERT was started after visit 1 and one year before visit 2.

FIG. 6: Correlation of alpha-galactosidase A activity in leucocytes (nmol/min/mg protein) with mean percentage of globotriaosylceramide (Gb3) positive peripheral blood mononuclear cells (PBMC) in men (A) and women (B) with Fabry disease (FD). A negative correlation was found for both genders (men: Spearman correlation coefficient: −0.451, p<0.05; women: Spearman correlation coefficient: −0.423, p<0.05).

FIG. 7: Bar graphs illustrate alpha-galactosidase A (α-GAL) activity and mean percentage of globotriaosylceramide (Gb3) positive peripheral blood mononuclear cells (PBMC) of men (M) and women (F) with Fabry disease (FD) carrying missense (MS) and nonsense (NS) mutations. In men (A) and women (B) with NS mutations, α-GAL activity was lower compared to those with MS mutations. This was reciprocal to the mean percentage of Gb3 positive PBMC, which was higher in men (A) and women (B) with NS mutations compared to those with MS mutations, ***p<0.01, *p<0.05.

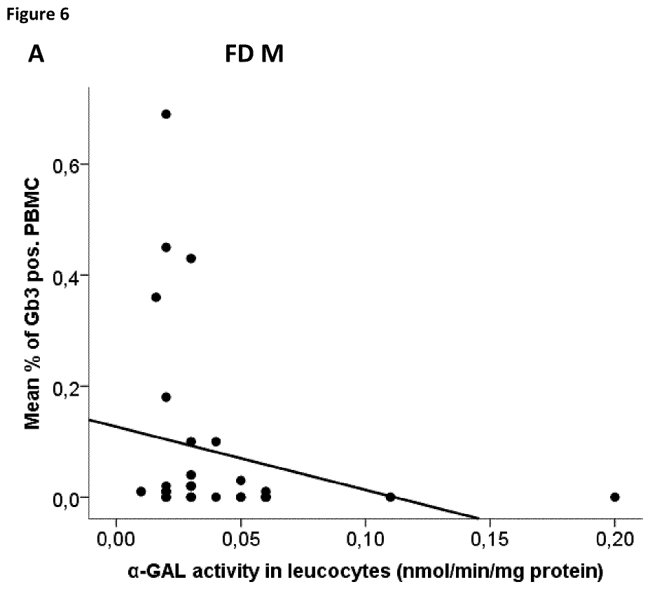

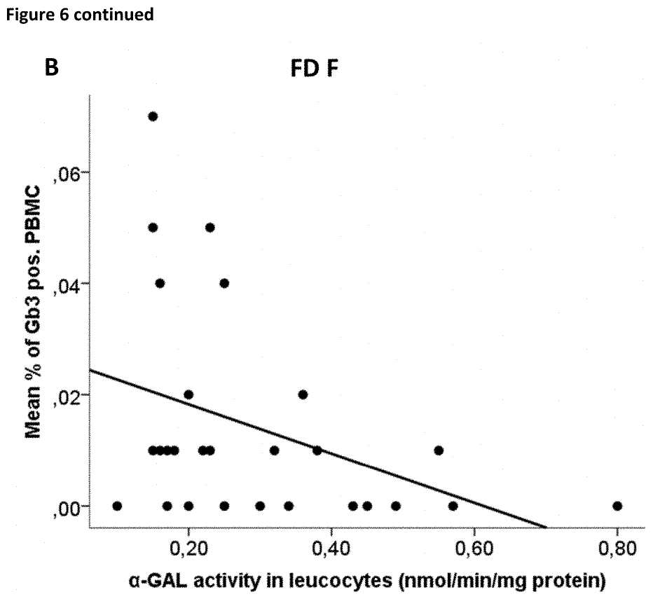

FIG. 6: Bar graphs illustrate plasma lyso-Gb3 levels in male (M) and female (F) patients with Fabry disease (FD) carrying missense (MS) and nonsense (NS) mutations. In men (A) and women (B), lyso-Gb3 was higher in patients with NS mutations compared to those carrying MS mutations. When investigating plasma lyso-Gb3 levels and patients' mean percentage of globotriaosylceramide (Gb3) positive peripheral blood mononuclear cells (PBMC), a positive correlation was found in men (C; Spearman correlation coefficient: 0.705, p<0.001) and women (Spearman correlation coefficient: 0.499, p<0.01) for high lyso-Gb3 levels with high numbers of Gb3 positive PBMC. ***p<0.001, n.s.: not significant.

FIG. 9: Bar graphs illustrate the plasma globotriaosysphingosine (tyso-Gb3) levels of men (M) and women (F) with Fabry disease (FD) carrying classical (CL) mutations and with or without enzyme replacement therapy (ERT). ERT did not influence lyso-Gb3 levels.

FIG. 10: Receiver operating characteristic (ROC) curves are shown for the number of globotriaosylceramide (Gb3) positive peripheral blood mononuclear cells (PBMC) in men (Sensitivity: 91%, specificity 69%, AUC: 0-797, SE: 0.75, Asymptotic sig.: p=0.001. Asymptotic 95% conf, interval: 0.650-0.943) (A) and women (Sensitivity: 91%, specificity 67%, AUC: 0-790, SE: 0.83. Asymptotic sig.: p=0.003. Asymptotic 95% conf. Interval: 0.628-0.952) (B) with Fabry disease (FD) and carrying classical mutations (patients without treatment and patients having received enzyme replacement therapy >8 days before) compared to healthy controls. The relative frequency of “true positive” values on the y-axis (i.e. sensitivity) is plotted against the relative frequency of “true negative” values on the x-axis (i.e. 1−sensitivity),

FIG. 11: Blood smear prepared using 10 μl venous whole blood of a patient with Fabry disease and a healthy control (B) immunoreacted with antibodies against Gb3. Yellow arrows indicate some of the many cellular Gb3 deposits. No Gb3 deposits are visible in the sample of the healthy control. Scale bar: 50 μm.

FIG. 12: Blood smear prepared using a drop of finger stick capillary blood of a patient with Fabry disease and a healthy control (B) immunoreacted with antibodies against Gb3. Yellow arrow indicates a cellular Gb3 deposition. No Gb3 deposits are visible in the sample of the healthy control. Scale bar 50 μm. Investigation of blood smears of a male Fabry patient carrying a classical Fabry mutation using the commercial antibody (A) and Shiga toxin (B). While hardly any deposits of Gb3 are seen using the antibody, Shiga toxin reveals dense accumulation of Gb3 in blood cells.

FIG. 13: The scheme summarizes potential ways how to transfer our idea of detecting Gb3 in blood cells to a test kit for self-administration, named FABRYSTIX.

FIG. 14: Buccal smear prepared using a buccal swab of a patient with Fabry disease immunoreacted with antibodies against Gb3. Several epithelial cells are visible with a blue nucleus and one shows a green signal potentially indicating Gb3 deposition. Scale bar: 50 μm.

FIG. 15: The Figure shows the genetic distribution in the study population. Abbreviations: CL: classical mutation (i.e. the mutation is known to be associated with classical symptoms and signs of FD); NCL: non-classical mutation (i.e. the mutation is associated with late onset or predominant involvement of one organ).

### EXAMPLES

The following examples illustrate the invention. These examples should not be construed as to limit the scope of this invention. The examples are included for purposes of illustration and the present invention is limited only by the claims.

Gb3 Detection in Peripheral Blood Mononuclear Cells (PBMCs)

The inventors investigated 67 consecutive adult FD patient's age 217 years who reported at the Wurzburg Fabry Center for Interdisciplinary Therapy (FAZIT) between 2014 and 2017. The group consisted of 37 men (median age 47 years, range 17-67 years) and 30 women (median age 52 years, range 19.78 years). Additionally, 52 healthy volunteers were recruited as controls. The control group consisted of 26 men (median age 52 years, range 24-77 years) and 26 women (median age 50, range 27-76 years). For the extraction of PBMCs venous blood was obtained in 8×9 ml EDTA containing monovettes. From these blood samples PBMCs were isolated following the protocol described in detail below (Example 1). The PBMCs obtained were then immunoreacted following the protocol also described in Example 1. The results of the immunoreaction were analyzed using a fluorescence microscope (Axiophot 2 microscope. Zeiss, Jena, Germany) that was equipped with a CCD camera (Visitron Systems, Tuchheim) and SPOT Advanced Software (Windows Version 4.5. Diagnostic instruments, Inc, Sterling Heights, USA).

The protocol for isolation of PBMC comprises the following steps: venous blood is collected in 8×9 ml EDTA-containing monovettes, after mixing, the content of monovettes is transferred into clean Falcon tubes, more precisely, the content of 2 monovettes is used to fill a 50 ml Falcon tube, to reach a maximum volume of 17.5 mL Subsequently, the same volume of 1×PBS buffer (i.e. maximum 17.5 ml) is added to each prepared Falcon and mixed gently. 15 ml of Lymphoprep (RT) are then added into 4 new 50 mi Falcon tubes, and the content of one Falcon tube each (containing the blood-PBS buffer mixture) is included very slowly to the Lymphoprep, in a way that the two solutions do not mix up. These Falcon tubes are then centrifuged for 20 min at 20° C. and 1800 U without breaks. During the centrifugation step, a white ring will form in the each Falcon tube. Two of these rings are to be carefully and entirely collected with a plastic pipette and put in a new 50 ml Falcon tube. Each falcon tube containing said rings is then filled up to 50 ml with 1×PBS and centrifuged for 12 min at 4° C. and 1400 U with breaks. After centrifugation the supernatant is removed up to approximately 5 mi, the cell pellets are resuspended with a sterile pipette and pooled in a new 50 ml Falcon tube, which is again filled up to 50 ml with 1×PBS, and mixed well. The new Falcon tubes are centrifuged for 2 min at 4° C. and 1400 U with breaks, the supernatant is removed with a pipette up to 200 μl. 9.8 ml 1×PBS is added and used to resolve the pellet with a sterile pipette. After pellet resolution, 10 μl trypan blue is added in one well of a plastic well-plate and mixed with 10 μl of the resolved cells. 10 μl of this mixture is added to a Neubauer Improved chamber and 5 squares are diagonally counted (for example: 10⁸ cells counted≙10.8×10⁷ cells/ml). The rest of the resolved pellets is again centrifuged 10 min at 4° C. and 1400 U with breaks, the supernatant is removed with a pipette. The cells are stored in 1 ml storage medium per 1×10⁷ cells at −80° C. OR resolve cells with a dilution of 1×10⁶ cells/ml in 1×PBS when directly going on with the staining protocol below.

A protocol recently developed for PBMCs staining comprises the following steps: On the first day. 1×10⁵ cells in 25 μl 1×PBS are pipetted as a drop on a glass slide and dried for 2 hours, the cells are then fixed for 10 min at room temperature (RT) with 4% PFA, and washed 3 times for 5 minutes with 1×PBS. Subsequently cells are permeabilized for 5 minutes at room temperature (RT) with 0.3% Triton X-100 in PBS (=0.3% PBST). The solution is allowed to drop off well (i.e not washed), and cells are blocked for 1 h at RT with 10% BSA/PBS. Again the solution is allowed to drop off well (i.e. not washed). The Gb3-antibody is diluted (see below) in 0.01% PBST to 1:250 and pipetted on the cells (final volume: 50-75 μl). Cells are incubated with the antibody at 4° C. over night in humid chamber. On the second day, cells are washed 3 times for 5 minutes with PBS. The secondary antibody (donkey-anti-mouse Alexa Fluor (Nr. Z3)) is diluted 1:150 (a final dilution 1:300) in 0.01% PBST pipetted on cells and incubated for 1 h at RT. The cells are then washed 1×5 min with 1×PBS, Incubated for 10 minutes with DAPI diluted 10.000 in 1×PBS, washed 3 times for 5 min with 1×PBS and covered with Aqua Poly/Mount. A negative control is also prepared by incubating the cells with blocking solution over-night instead of primary antibody and then the regular protocol and incubation with secondary antibody is performed.

Clinical Characteristics of the Patient Population

Table 1 provides individual data.

TABLE 1

| | | | | | | | Days | Mean % | α-GAL | |
|---|---|---|---|---|---|---|---|---|---|---|
| | | | | | | Duration | since | of Gb3 | activity | Lyso- |
| | | | CL, | MS, | | of ERT | last | pos. | (nmol/min/ | Gb3 |
| # | Gender | Age | NCL | NS | Genotype | (years) | ERT | PBMC | mg protein) | (ng/ml) |
| 1 | M | 21 | CL | NS | W349X | none | none | 0.0113 | 0.01 | 27.0 |
| 2 | M | 44 | CL | NS | c.679 C > T//R227X | none | none | 0.1810 | 0.02 | 25.2 |
| 3 | M | 46 | CL | NS | c. 1196G > A//W399X | none | none | 0.4497 | 0.02 | 29.2 |
| 4 | M | 37 | CL | NS | c.1029_1030 del TC fsX30 | 8 | 11 | 0.6880 | 0.02 | 5.2 |
| 5 | M | 20 | CL | NS | c. 363 delT//A121fs*8 | 7 | 12 | 0.0263 | 0.05 | 4.8 |
| 6 | M | 22 | CL | NS | c. 363 delT//A121fs*8 | 7 | 13 | 0.0017 | 0.05 | 7.2 |
| 7 | M | 57 | CL | NS | c. 611 G > A//W204X | 4 | 13 | 0.4337 | 0.03 | 9.0 |
| 8 | M | 32 | CL | NS | c.1069 C > T//p. Q357X | 6 | 3 | 0.0103 | n.d. | 6.9 |
| | | | | | (p.Gln357X) | | | | | |
| 9 | M | 47 | CL | NS | c. 1196G > A//W399X | 15 | 4 | 0.0037 | 0.03 | 12.7 |
| 10 | M | 40 | CL | NS | W349X | 11 | 4 | 0.0197 | 0.02 | 75.4 |
| 11 | M | 17 | CL | NS | c.993_994 ins A (fsX338) | 7 | 8 | 0.0060 | 0.02 | 170.0 |
| 12 | M | 47 | CL | MS | c.708G > C//p.W236C | none | none | 0.5540 | 0.06 | 27.7 |
| 13 | M | 65 | CL | MS | c.720G > C//p.K240N | none | none | 0.0153 | 0.03 | 11.4 |
| 14 | M | 51 | CL | MS | c.406G > C (D136H) | none | none | 0.3600 | 0.02 | 22.1 |
| 15 | M | 53 | CL | MS | c.404 C > T//A135V | 8 | 14 | 0.0010 | 0.20 | 24.6 |
| 16 | M | 48 | CL | MS | c.408 T > A//D136E | 11 | 15 | 0.0053 | 0.02 | 27.9 |
| 17 | M | 52 | CL | MS | c.102 G > A//E341K | 14 | 18 | 0.0083 | 0.02 | 14.7 |
| 18 | M | 44 | CL | MS | c.845C > T//T282I | 7 | 1 | 0.0020 | 0.02 | 7.3 |
| 19 | M | 18 | CL | MS | Q321H (c.963G > C) + | 3 | 2 | 0.0417 | 0.03 | 22.6 |
| | | | | | D322N(c.964G > A) | | | | | |
| 20 | M | 33 | CL | MS | c.508 G > A//D170N | 11 | 6 | 0.0007 | 0.02 | 152.0 |
| 21 | M | 35 | CL | MS | c.486G > T//W162C | 2 | 6 | 0.0077 | n.d. | n.d. |
| 22 | M | 50 | CL | MS | c.103 G > A//G35R | 10 | 8 | 0.0080 | n.d. | 14.4 |
| 23 | M | 37 | CL | consensus | IVS2 + 1 (G > A) | 0.1 | 13 | 0.1040 | 0.03 | 109.0 |
| | | | | splice | | | | | | |
| | | | | mutation | | | | | | |
| 24 | M | 48 | CL | consensus | IVS3 + 1 G > A | 16 | 21 | 0.0050 | n.d. | 14.6 |
| | | | | splice | | | | | | |
| | | | | mutation | | | | | | |
| 25 | M | 38 | CL | consensus | IVS6 − 10G > A, c.1000 − | 4 | 6 | 0.0963 | 0.04 | 112.0 |
| | | | | splice | 10G > A | | | | | |
| | | | | mutation | | | | | | |
| 26 | M | 24 | NC | MS | c.416 A > G//N139S | none | none | 0.0100 | 0.06 | 44.3 |
| 27 | M | 44 | NC | MS | c.427 G > A//A143T | none | none | 0.0007 | 0.11 | 34.4 |
| 28 | M | 21 | NC | MS | c.416 A > G//N139S | 4 | 12 | 0 | 0.06 | 102.0 |
| 29 | M | 50 | NC | MS | c.1208 del AAG | 12 | 12 | 0 | 0.03 | 46.8 |
| 30 | M | 60 | NC | MS | c.386 T > C//L129P | 9 | 15 | 0.0007 | 0.02 | 23.1 |
| 31 | M | 34 | NC | MS | c.386 T > C//L129P | 7 | ERT | 0.0177 | 0.03 | 70.0 |
| 32 | M | 63 | NC | MS | 644 A > G (N215S) | 2 | 6 | 0.0007 | 0.06 | 27.2 |
| 33 | M | 67 | NC | MS | c.644 A > G (N215S) | 2 | 7 | 0 | n.d. | 4.8 |
| 34 | M | 62 | NC | MS | c.644 A > G (N215S) | 5 | 9 | 0.0017 | 0.05 | 7.2 |
| 35 | M | 62 | NC | MS | c.644 A > G (N215S) | none | none | 0.0043 | 0.05 | 9.0 |
| 36 | M | 51 | NC | MS | c.644 A > G (N215S) | none | none | 0 | 0.06 | 6.9 |
| 37 | M | 66 | NC | MS | c.644 A > G (N215S) | none | none | 0.0040 | 0.04 | 12.7 |
| 38 | F | 52 | CL | NS | c. 1196G > A//W399X | none | none | 0.0053 | 0.15 | 9.4 |
| 39 | F | 22 | CL | NS | c.404 C > T//A135V | none | none | 0.0077 | 0.18 | 5.7 |
| 40 | F | 28 | CL | NS | c.1069 C > T//p. Q357X | none | none | 0.0500 | 0.15 | 9.5 |
| | | | | | (p.Gln357X) | | | | | |
| 41 | F | 35 | CL | NS | c.718-719delAA (fs248X) | none | none | 0.0100 | 0.17 | 19.6 |
| 42 | F | 19 | CL | NS | c.993_994 ins A (fs X 338) | none | none | 0.0663 | 0.15 | 7.4 |
| 43 | F | 41 | CL | NS | c.993_994 ins A (fs X 338) | 1 | 7 | 0.0203 | 0.20 | 7.0 |
| 44 | F | 44 | CL | NS | c.363delT//A121fs*8 | 4 | 12 | 0.0017 | 0.25 | 10.8 |
| 45 | F | 26 | CL | MS | c.404 C > T//A135V | none | none | 0.0387 | 0.16 | 17.0 |
| 46 | F | 41 | CL | MS | c.874G > C (p. | none | none | 0.0130 | n.d. | 9.7 |
| | | | | | Ala292Pro)/A292P | | | | | |
| 47 | F | 46 | CL | MS | C.860G > C// | none | none | 0.0473 | 0.23 | 17.4 |
| | | | | | p.Trp287Ser | | | | | |
| 48 | F | 63 | CL | MS | c.484 T > G//W162G | none | none | 0.0103 | 0.32 | 7.8 |
| 49 | F | 53 | CL | MS | c.404 C > T//A135V | none | none | 0.0163 | 0.36 | 17.1 |
| 50 | F | 53 | CL | MS | c.1025 G > T//R342L | 12 | 22 | 0 | 0.34 | 4.9 |
| 51 | F | 54 | CL | MS | Q321H (c.963G > C) + | 0.1 | 1 | 0.0007 | 0.17 | 13.3 |
| | | | | | D322N(c.964G > A) | | | | | |
| 52 | F | 52 | CL | MS | c.404 C > T//A135V | 3 | 7 | 0.0003 | 0.10 | 13.0 |
| 53 | F | 78 | CL | MS | c.103 G > A//G35R | 9 | 8 | 0.0133 | 0.16 | 12.1 |
| 54 | F | 56 | CL | MS | c.1025 G > T//R342L | 12 | 8 | 0.0003 | 0.43 | 5.3 |
| 55 | F | 63 | NC | NS | c.1221 del A | none | none | 0.0380 | 0.22 | 17.8 |
| 56 | F | 57 | NC | NS | c.756 or 757 del A, | none | none | 0.0087 | 0.38 | 25.3 |
| | | | | | fs268X | | | | | |
| 57 | F | 49 | NC | MS | c.386 T > C//L129P | 4 | ERT | 0.0083 | 0.22 | 9.5 |
| 58 | F | 52 | NC | MS | c.973 G > A//G325S + | none | none | 0.0023 | 0.20 | 8.3 |
| | | | | | polymorphisms | | | | | |
| 59 | F | 75 | NC | MS | c.427 G > A//A143T | none | none | 0.0013 | 0.49 | 1.1 |
| 60 | F | 59 | NC | MS | c.644 A > G (N215S) | none | none | 0.0003 | n.d. | 1.7 |
| 61 | F | 75 | NC | MS | c.644 A > G (N215S) | none | none | 0.0073 | 0.55 | 1.5 |
| 62 | F | 23 | NC | MS | c.937G > T//D313Y | none | none | 0.0020 | 0.80 | 0.8 |
| 63 | F | 19 | NC | MS | c.937G > T//D313Y | none | none | 0 | n.d. | 0.5 |
| 64 | F | 45 | NC | MS | c.937G > T, p. | none | none | 0.0100 | 0.23 | 0.6 |
| | | | | | Asp313Tyr (D313Y) | | | | | |
| 65 | F | 57 | NC | MS | c.937G > T (D313Y) | none | none | 0 | 0.57 | 0.6 |
| 66 | F | 54 | NC | MS | c.1196G > C (bet) | 4 | 5 | 0 | 0.30 | 0.7 |
| | | | | | p.W399S + IVS2 − 81_77 | | | | | |
| | | | | | homo, −10C > T | | | | | |
| | | | | | homoz, IVS4 − 16 A > G | | | | | |
| | | | | | homoz, IVS6 − 22 C > T | | | | | |
| | | | | | homoz | | | | | |

α-GAL: α -galactosidase A, CL: classical mutation, ERT: enzyme replacement therapy, F: female, Gb3: globotriaosylceramide, M: male, MS: missense; NS: non-sense, NCL: non-classical mutation, n.d.: no data.
*Patients (mother and son) refused sharing the date of the last ERT.

Table 2 gives a baseline data of the patient population:

TABLE 2

| | [symbol] | [symbol] |
|---|---|---|
| [symbol] | [symbol] | [symbol] |
| [symbol] | [symbol] | [symbol] |
| [symbol] | | |
| [symbol] | | |
| [symbol] | | |
| [symbol] | [symbol] | [symbol] |
| [symbol] | | |
| [symbol] | | |
| [symbol] | [symbol] | [symbol] |
| [symbol] | [symbol] | [symbol] |
| [symbol] | [symbol] | [symbol] |
| [symbol] | [symbol] | [symbol] |
| [symbol] | | |
| [symbol] | | |
| [symbol] | [symbol] | [symbol] |
| [symbol] | | |
| [symbol] | | |
| [symbol] | | |

| | M | F |
|---|---|---|
| N | 37 | 30 |
| Median age | 47 (17-67) | 52 (19-78) |
| (range) | | |
| (years) | | |
| Genotype | | |
| CL | 25 | 17 |
| Type of | | |
| mutation | | |
| NS | 11 | 8 |
| MS | 23 | 21 |
| other^{a} | 3 | 1 |
| Number of | 26/37 (70%) | 9/30 (30%) |
| patients | | |
| On ERT | | |
| Median time | 7 (0.1-16) | 4 (0.1-12) |
| since ERT | | |
| (range) | | |
| (years) | | |

CL, classical mutation; ERT, enzyme replacement therapy; F, female; M, male; MS, missense mutation; NCL, nonclassical mutation; NS, nonsense mutation.
^{a}Intronic consensus splice nutations.

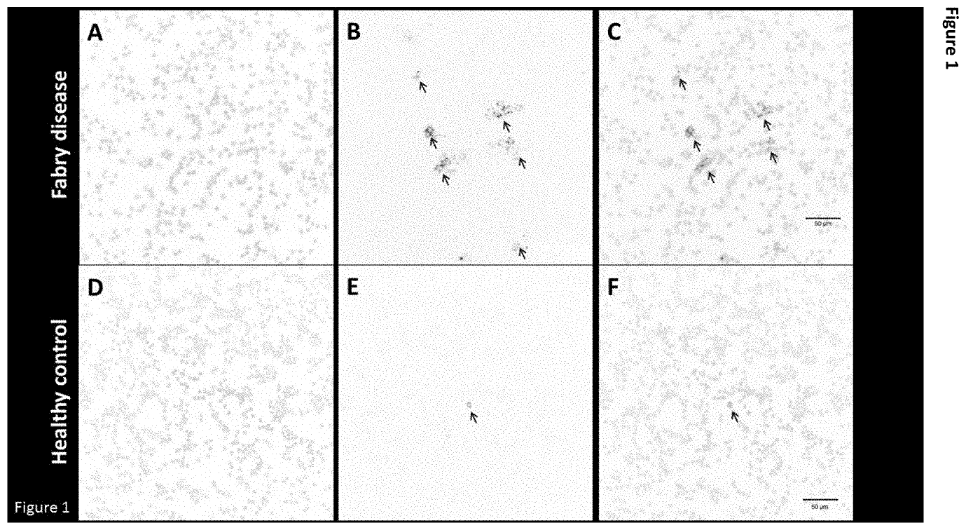

Among men with FD, n=25 had a mutation likely leading to a classic phenotype (i.e. the mutation is known to be associated with typical symptoms and signs of FD) and n=12 had a mutation likely leading to a non-classic phenotype (i.e. the mutation is associated with late onset or predominant involvement of one organ) (van der Tol et al., 2014 JIMD Rep. 17, 83-90). Among women with FD, n=17 had a classical and n=12 a non-classical genotype. Cases were allocated after individual cross-check of the genotype using https://lih16.u.hpc.mssm.edu/pipeline/js/dbFabry/ Mutation.html#. Supplementary FIG. 1 summarizes the genotypic distribution of the study cohort. 19/25 men and 7/17 women with classical mutation and 7/12 men and 2/12 women with non-classical mutation were on ERT (15 men on agalsidase-beta, 10 on agalsidase-alpha, 1 switched from agalsidase-beta to agalsidase-alpha and back; 3 women on agalsidase-beta, 6 on agalsidase-alpha). The median time on ERT was 7 years (0.1-16) in men and 4 years (0.1-12) in women.

Gb3 Positive PBMC can be Visualized and are More Frequent in Men and Women with FD than in Healthy Controls

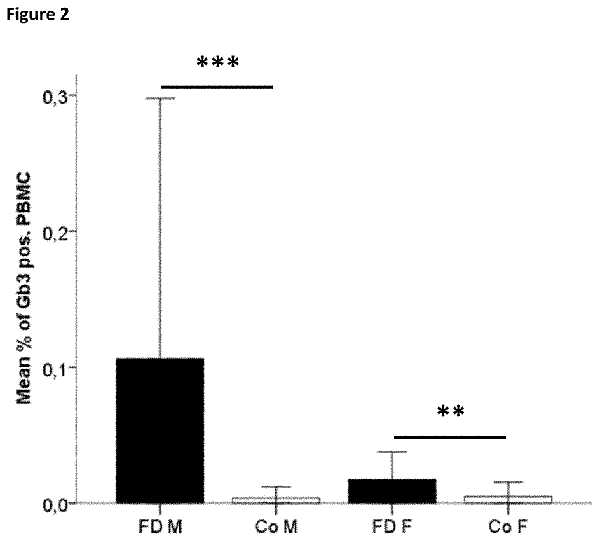

Gb3 deposits were distinctly visible in the cytosol of PBMC of patients with FD and to a much lesser extent in healthy controls (FIG. 1). The mean percentage of Gb3 positive PBMC was higher in men (p<0.001) and women (p<0.01) with FD compared to male and female controls (FIG. 2).

Men and Women Carrying Classical FD Mutations have a Higher Number of Gb3 Positive PBMC, while Patients with Non-Classical Mutations do not Differ from Controls

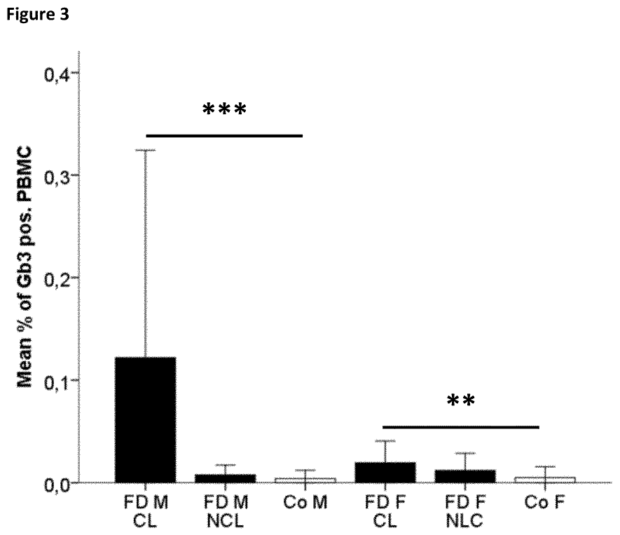

The mean percentage of Gb3 positive PBMC was sixteen-fold higher in men carrying a classical mutation (0.08) compared to healthy men (0.005; p<0.001), while men carrying a non-classical mutation were not different from male controls (FIG. 3). Similarly, women carrying a classical mutation had four-fold higher load of Gb3 positive PBMC (0.02) than healthy women (0.005; p<0.01), while those with non-classical mutations were not different from controls (FIG. 3).

Number of Gb3 Positive PBMC Temporarily Decreases Under ERT

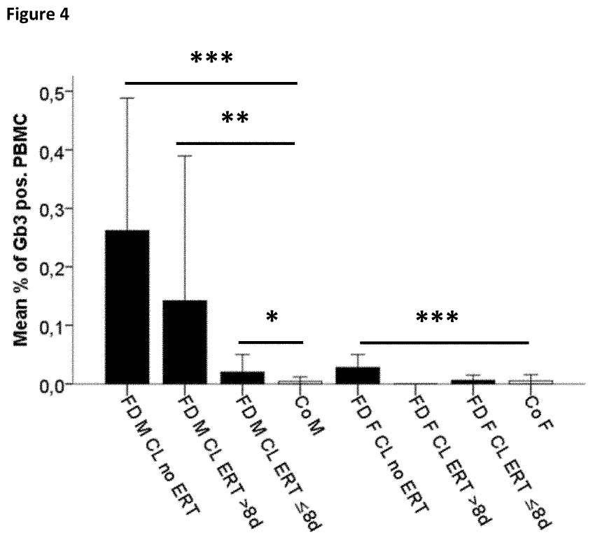

The Inventors next investigated, if the number of Gb3 positive PBMC changes with ERT. In men carrying classical mutations, the number of Gb3 positive PBMC consecutively decreased with ERT: the mean percentage of Gb3 positive PBMC was highest in untreated men (p<0.001), lower in those with treatment >8 days before (p<0.01), and close to normal in men with treatment up to eight days before blood withdrawal compared to healthy men (p<0.05; FIG. 4). The mean percentage of Gb3 positive PBMC was higher in untreated men carrying classical mutations compared to those under ERT and carrying classical mutations (p<0.05). In female FD patients with classical mutations, the mean percentage of Gb3 positive PBMC was also highest in untreated women (p<0.001 compared to healthy female controls: FIG. 4). No intergroup difference was found when comparing women with treatment >8 days (n=1) and ≤8 days (n=5) before ERT compared to controls, which may be due to the low number of subjects per subgroup, however, the number of Gb3 positive PMBC was lower in treated women compared to untreated ones (p<0.05). Among men and women carrying non-classical mutations, only n=7 respectively n=2 patients were on ERT, and the number of Gb3 positive PBMC was not different from healthy controls each (data not shown).

Load of Gb3 Positive PBMC Decreased Under Long-Term ERT

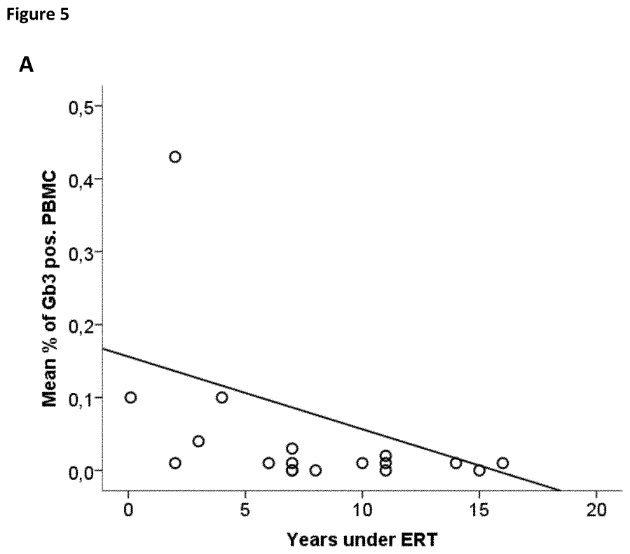

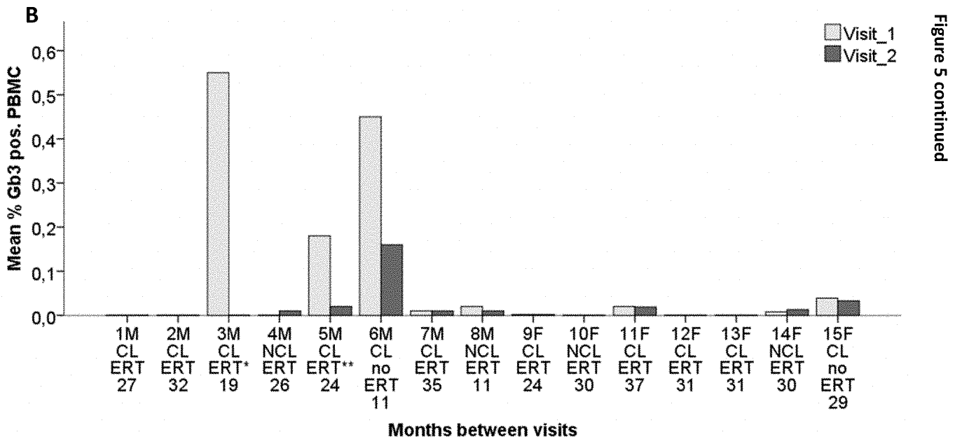

The load of Gb3 positive PBMC decreased with total duration of treatment in men with FD and carrying classical mutations (Spearman correlation coefficient −0.457, p<0.05. FIG. 5A). No correlation was found between the mean percentage of Gb3 positive PBMC and age when investigating untreated male and female FD patients with classical and non-classical mutations (data not shown).

Of n=15 patients (8 men, B with classical mutation, 5 on ERT at both visits; 7 women, 5 with classical mutation, 6 on ERT at both visits) the inventors obtained a second blood sample after a median time of 25 months in men (11-35) and 30 months in women (24-37). In all patients receiving ERT, the mean percentage of Gb3 positive PBMC was low at both visits. One male patient started ERT Just after visit 1 and 19 months before visit 2 (#3 in FIG. 5B), a second male patient started ERT a year after visit 1 and 12 months before visit 2 (#5 in FIG. 58): both showed a drop of Gb3 load at visit 2. In contrast, the mean percentage of Gb3 positive PBMC remained high in a male patient who had stopped ERT six years before visit 1 (#6 in FIG. 5B) and a female patient who had not yet initiated treatment (#15 in FIG. 58).

The Mean Percentage of Gb3 Positive PBMC Correlates with α-GAL Activity and with Lyso-Gb3

The inventors next investigated, if α-GAL activity is reflected by the number of Gb3 positive PBMC. The median α-GAL activity measured in leucocytes was 0.03 nmol/min/mg protein in men (0.01-0.2) and 0.23 nmol/min/mg protein in women (0.1-0.8). We found a negative correlation in men (Spearman correlation coefficient: −0.451, p<0.05) and women with FD (Spearman correlation coefficient −0.423, p<0.05) with lower α-GAL activity being associated with a higher mean percentage of Gb3 positive PBMC (FIG. 6).

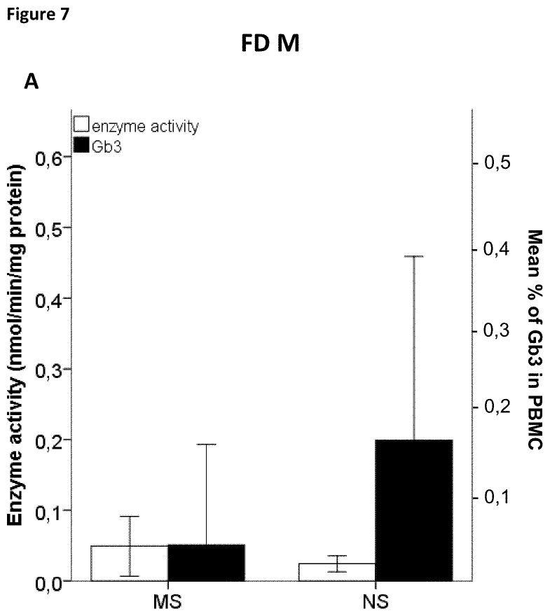

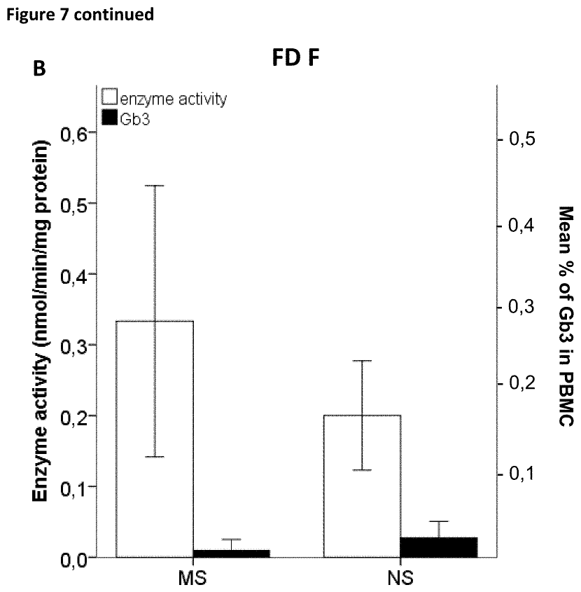

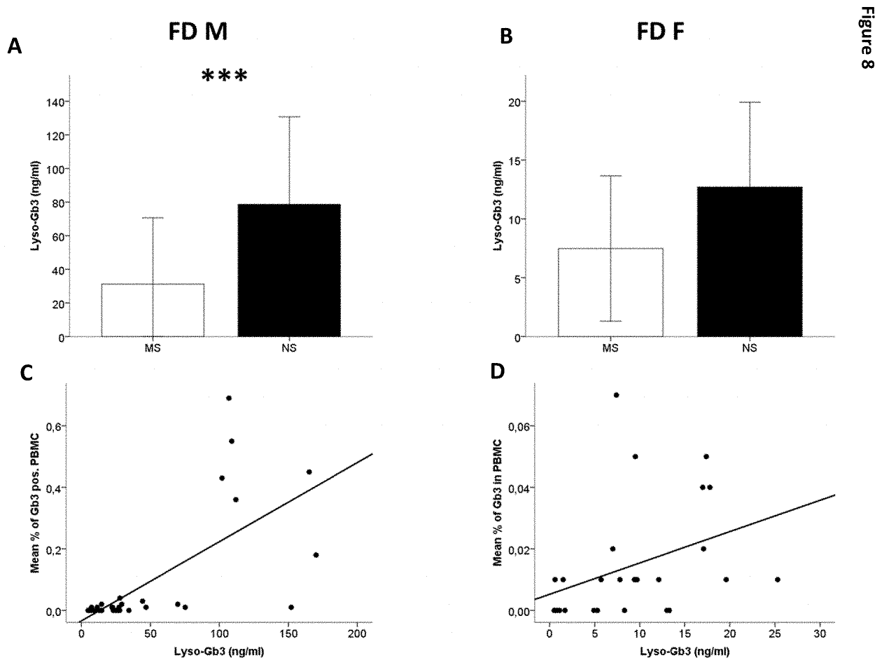

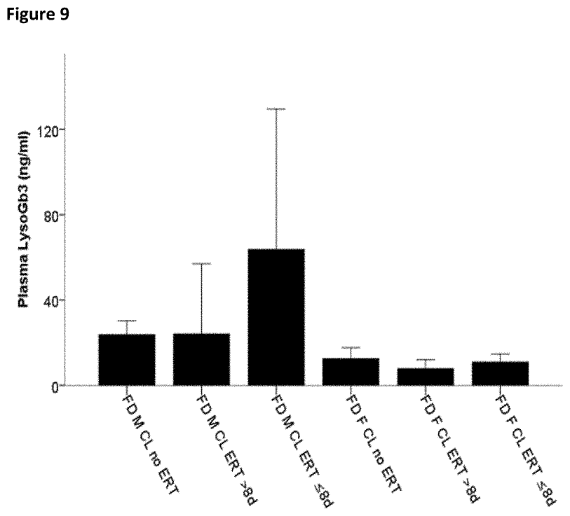

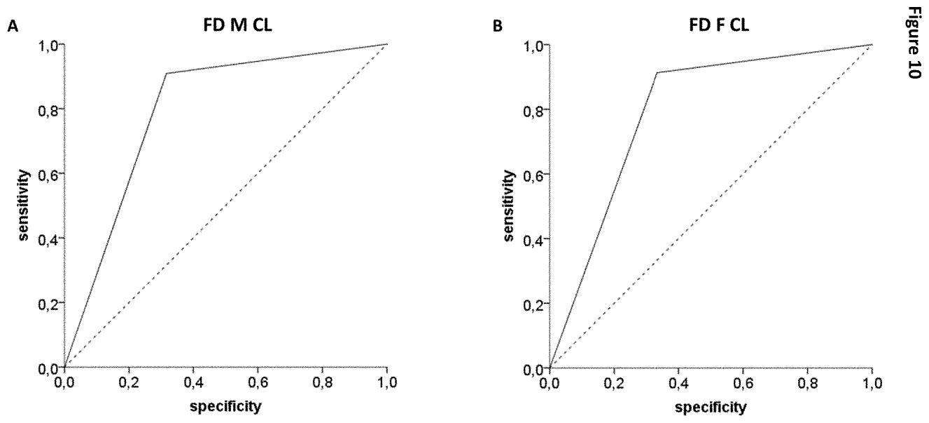

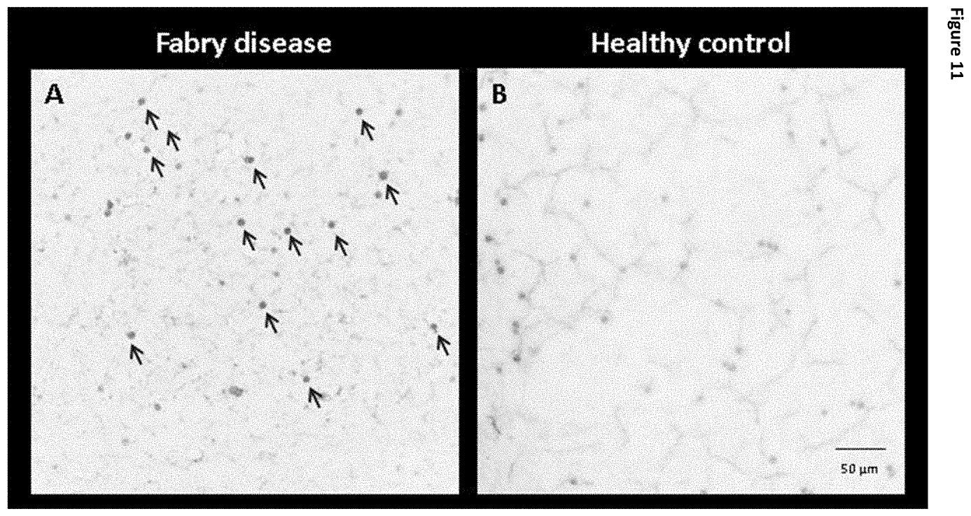

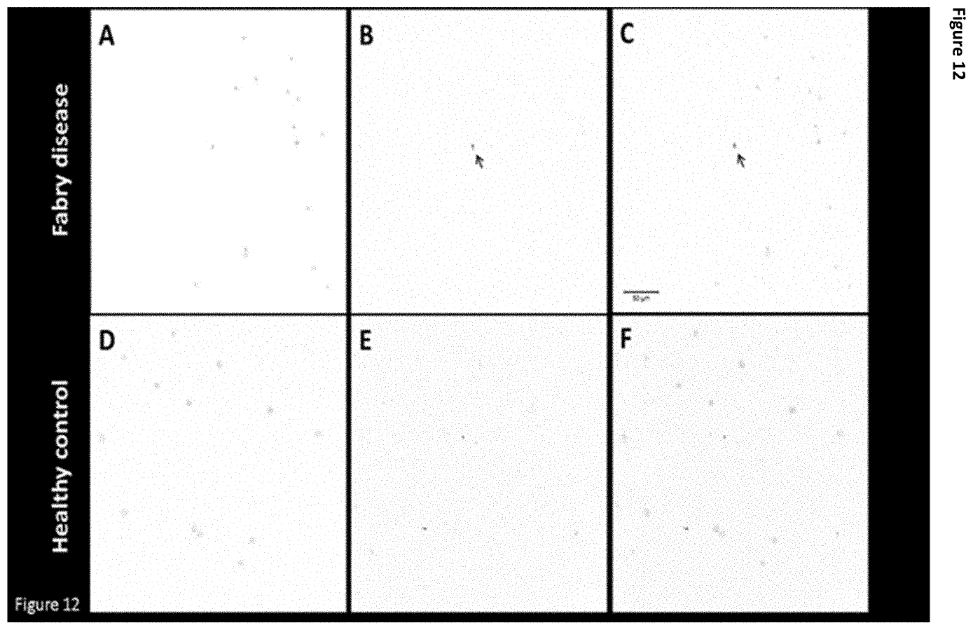

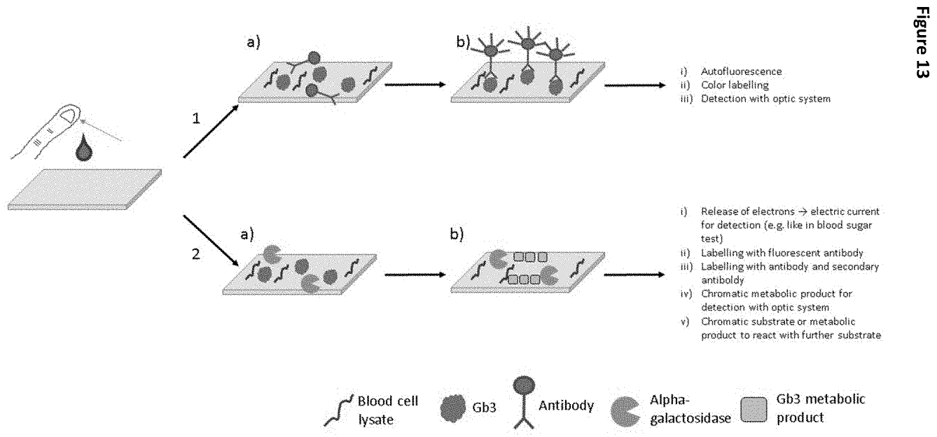

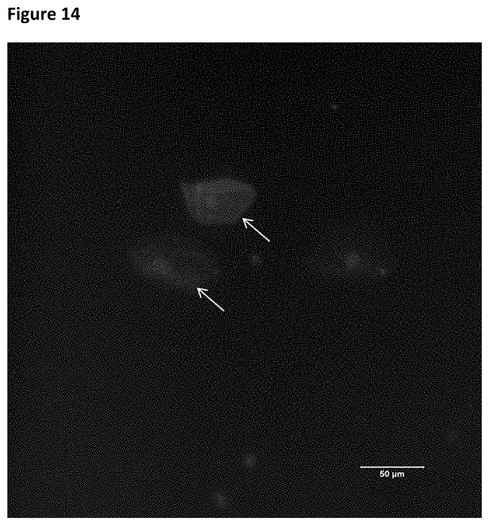

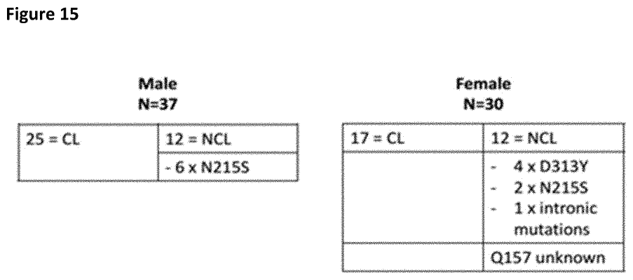

When stratifying the entire patient cohort for nonsense and missense mutation carriers, the inventors found a higher mean percentage of Gb3 positive PBMC in men (p<0.05) and women (p<0.01) with nonsense mutations (FIG. 7), which was reciprocal to α-GAL activity: men (p<0.05) and women (p<0.05) carrying nonsense mutations had lower α-GAL activity compared to men and women with missense mutations (FIG. 7). Accordingly, men (p<0.001) and women (n.s.) with nonsense mutations had higher plasma levels of lyso-Gb3 than patients carrying missense mutations (FIG. 8A, B), the inventors found a strong positive correlation in men (Spearman correlation coefficient: 0.705, p<0.001) and women with FD (Spearman correlation coefficient: 0.499, p<0.01) of lyso-Gb3 levels and mean percentage of Gb3 positive PBMC (FIG. 8C, D).

The cohort contained eleven families with two (n=8), three (n=2), and five (n=1) family members. Mean percentages of Gb3 positive PBMC individually varied between family members (Table 3).

Table 3

| | | | | | | | | | | |
|---|---|---|---|---|---|---|---|---|---|---|
| | | | | | | | Days | Mean % | α-GAL | |
| | | | | | | Duration | since | of Gb3 | activity | Lyso- |
| | | | CL, | MS, | | of ERT | last | pos. | (nmol/min/ | Gb3 |
| # | Gender | Age | NCL | NS | Genotype | (years) | ERT | PBMC | mg protein) | (ng/ml) |

Family 1

| | | | | | | | | | | |
|---|---|---|---|---|---|---|---|---|---|---|
| 19 | M | 18 | CL | MS | c.963G > C, Q321H + c.964G > A, | 3 | 2 | 0.0417 | 0.03 | 22.6 |
| | | | | | D322N | | | | | |
| 51 | F | 54 | CL | MS | c.963G > C, Q321H + c.964G > A, | 0.1 | 1 | 0.0007 | 0.17 | 13.3 |
| | | | | | D322N | | | | | |

Family 2

| | | | | | | | | | | |
|---|---|---|---|---|---|---|---|---|---|---|
| 8 | M | 32 | CL | NS | c.1069 C > T, p. Q357X, | 6 | 3 | 0.0103 | n.d. | 6.9 |
| | | | | | P.Gln357X | | | | | |
| 40 | F | 28 | CL | MS | c.1069 C > T, p. Q357X, | none | none | 0.0500 | 0.15 | 9.5 |
| | | | | | P.Gln357X | | | | | |

Family 3

| | | | | | | | | | | |
|---|---|---|---|---|---|---|---|---|---|---|
| 50 | F | 53 | CL | MS | c.1025 G > T, R342L | 12 | 22 | 0 | 0.34 | 4.9 |
| 54 | F | 56 | CL | MS | c.1025 G > T, R342L | 12 | 8 | 0.0003 | 0.43 | 5.3 |

Family 4

| | | | | | | | | | | |
|---|---|---|---|---|---|---|---|---|---|---|
| 22 | M | 50 | CL | MS | c.103 G > A, G35R | 10 | 8 | 0.0080 | n.d. | 14.4 |
| 53 | F | 78 | CL | MS | c.103 G > A, G35R | 9 | 8 | 0.0003 | 0.16 | 12.1 |

Family 5

| | | | | | | | | | | |
|---|---|---|---|---|---|---|---|---|---|---|
| 31 | M | 34 | NCL | MS | c.386 T > C, L129P | 7 | ERT* | 0.0177 | 0.33 | 70.0 |
| 55 | F | 49 | NCL | MS | c.386 T > C, L129P | 4 | ERT* | 0.0083 | 0.22 | 9.5 |

Family 6

| | | | | | | | | | | |
|---|---|---|---|---|---|---|---|---|---|---|
| 9 | M | 47 | CL | NS | c. 1196G > A, W399X | 15 | 4 | 0.0037 | 0.03 | 12.7 |
| 38 | F | 52 | CL | NS | c. 1196G > A, W399X | none | none | 0.0053 | 0.15 | 9.4 |

Family 7

| | | | | | | | | | | |
|---|---|---|---|---|---|---|---|---|---|---|
| 1 | M | 21 | CL | NS | Exon 7, W349X | none | none | 0.0113 | 0.01 | 27.0 |
| 10 | M | 40 | CL | NS | Exon 7, W349X | 11 | 4 | 0.0197 | 0.02 | 75.4 |

Family 8

| | | | | | | | | | | |
|---|---|---|---|---|---|---|---|---|---|---|
| 26 | M | 24 | NCL | MS | c.416 A > G, N139S | none | none | 0.0100 | 0.06 | 44.3 |
| 28 | M | 21 | NCL | MS | c.416 A > G, N139S | 4 | 12 | 0 | 0.06 | 102.0 |

Family 9

| | | | | | | | | | | |
|---|---|---|---|---|---|---|---|---|---|---|
| 11 | M | 17 | CL | NS | c.993_9934 ins A, fsX 338 | 7 | 8 | 0.0060 | 0.02 | 170.0 |
| 42 | F | 19 | CL | NS | c.993_9934 ins A, fsX 338 | none | none | 0.0663 | 0.15 | 7.4 |
| 43 | F | 41 | CL | NS | c.993_9934 ins A, fsX 338 | 1 | 7 | 0.0203 | 0.20 | 7.0 |

Family 10

| | | | | | | | | | | |
|---|---|---|---|---|---|---|---|---|---|---|
| 5 | M | 20 | CL | NS | c. 363delT, A121fs*8 | 7 | 12 | 0.0263 | 0.05 | 4.8 |
| 6 | M | 22 | CL | NS | c. 363delT, A121fs*8 | 7 | 13 | 0.0017 | 0.05 | 7.2 |
| 44 | F | 44 | CL | NS | c. 363delT, A121fs*8 | 4 | 12 | 0.0017 | 0.25 | 10.8 |

Family 11

| | | | | | | | | | | |
|---|---|---|---|---|---|---|---|---|---|---|
| 15 | M | 53 | CL | MS | c.404 C > T, A135V | 8 | 14 | 0.0010 | 0.20 | 24.6 |
| 45 | F | 26 | CL | MS | c.404 C > T, A135V | none | none | 0.0387 | 0.16 | 17.0 |
| 52 | F | 52 | CL | MS | c.404 C > T, A135V | 3 | 7 | 0.0003 | 0.10 | 13.0 |
| 49 | F | 53 | CL | MS | c.404 C > T, A135V | none | none | 0.0163 | 0.36 | 17.1 |
| 39 | F | 22 | CL | NS | c.404 C > T, A135V | none | none | 0.0077 | 0.18 | 5.7 |

α-GAL: α -galactosidase A, CL: classical mutation, ERT: enzyme replacement therapy, F: female, Gb3: globotriaosylceramide, M: male, MS: missense; NS: non-sense, NCL: non-classical mutation, n.d.: no data.
*Patients (mother and son) refused sharing the date of their last ERT.

Gb3 Deposits in PBMC Reflect Treatment Effects, but Lyso-Gb3 does not

The inventors then investigated, if lyso-Gb3 reflects the effect of ERT. In contrast to our results obtained for mean percentage of Gb3 positive PBMC (Figure. 4), plasma lyso-Gb3 levels were not influenced by ERT (shown for untreated and treated men and women carrying classical mutations; (FIG. 9).

Gb3 Deposits in PBMC are of Diagnostic Value in Men and Women with FD

The sensitivity/specificity of the mean percentage of Gb3 positive PBMC for the detection of FD was 91%/69% in men and 91%/67% in women carrying classical FD mutations (untreated patients and patients having received ERT ≥8 d before) and when setting the cut-off value at 0 Gb3 positive PBMC (FIG. 10).

### Gb3 Visualization in Blood Smears

To facilitate the procedure of Gb3 visualization in blood samples, the inventors investigated if blood smears immunoreacted with antibodies against Gb3 would also allow the detection of Gb3 positive blood cells. The qualitative assessment of blood samples obtained from two FD patients and two healthy controls as 10 μl whole venous blood (FIG. 11) or few drops of finger stick capillary blood (FIG. 12) revealed that Gb3 deposits were also unequivocally visible in blood smear preparations. These results are further expected to improve when using Gb3 specific staining with e.g. its natural ligands such as shiga toxin instead of the commercial antibody.

A newly established protocol for staining Gb3 in blood smears comprises the following steps: On the first day, few drops of blood are placed on a glass slide, these drops are then thinly streaked/smeared out over the glass slide (over approx. 1.7 cm×3 cm area). The streak is then allowed to dry at room temperature for 30 minutes and subsequently is fixed for 10 minutes using 4% PFA at room temperature. The cells of the blood smear are then permeabilized with 0.3% triton X-100 in PBS (=0.3% PBST) at room temperature. The fixing solution is dropped off well (i.e. not washed) and the smear is blocked with 1 h at room temperature with 10% BSA/PBS. The blocking solution is allowed to drop off well (i.e not washed) and the smear is incubated with anti Gb-3 antibody (1:250 dilution) in 0.01% PBST and pipetted on the cells to a final volume of 50-75 μl. The smear is incubated with the antibody over night in humid chamber. On the second day, after the incubation cells are washed 3 times for 5 minutes with PBS. The secondary antibody (donkey-anti-mouse Alexa Fluor (Nr. Z3)) is diluted 1:150 (a final dilution 1:300) in 0.01% PBST, pipetted on the smear and incubated for 1 h at RT. The cells are then washed 1×5 min with 1×PBS, Incubated for 10 minutes with DAPI diluted 10.000 in 1×PBS, washed 3 times for 5 min with 1×PBS and covered with Aqua Poly/Mount. A negative control is also prepared by incubating the smear with blocking solution over-night instead of primary antibody and then the regular protocol and incubation with secondary antibody is performed.

Gb3 Visualization is Also Possible in Buccal Swabs

Another example shows the use of buccal epithelial cells for Gb3 detection. FIG. 14 shows the first result of Gb3 visualization in buccal epithelial cells, used to create a buccal swab then immunoreacted with antibodies against Gb3. Also in this case the inventors prove that Gb3 deposits are unequivocally visible in buccal swab preparations. Analogously to the blood smear preparations, these results are further expected to improve when using Gb3 specific natural ligands e.g. shiga toxin.

Materials Used in the Described Protocols:

The anti Gb3-antibodies used were the following commercial antibodies: Anti-Gb3 monoclonal antibody (M. Kotani et al. 1994 Biochem. Biophys. 310, 89); Anti-Gb3 monoclonal antibody, Cat #: A2506: company: TC (Tokyo Chemical Industry Co.) http://www.tcichemicals.com/eshop/deidelcommodity/A2506/

The buffers and mediums used were composed as follows: 1% PBS (0.137 M NaCl, 0.05 M NaH2PO4, pH 7.4); 4% PFA (Distilled water, HCl, 1 N NaOH, Paraformaldehyde, 1×PBS): Storage medium (50 ml heat inactivated fetal bovine serum, 40 ml RPIM without additives, 10 ml DMSO).

The composition of 10×PBS stock solution is depicted in Table 4 and the pH has been titrated to 6.7.

TABLE 4

| | 5 liters | 10 liters | 2 liters |
|---|---|---|---|
| NaCl | 400 g | 800 g | 160 g |
| KCl | 10 g | 20 g | 4 g |
| Na₂HPO₄ | 71 g | 142 g | 28.4 g |
| Na₂HPO₄ × 1H₂o | 69 g | 138 g | 27.6 g |

For the preparation of 1 liter of 4% PFA; heat up to approximately 60° C. 800 ml 1×PBS in a glass bottle on a heat plate under the hood, add 40 g paraformaldehyde and mix with a magnetic stirring rod. Add 1 N NaOH dropwise until the paraformaldehyde is completely dissolved. After cooing, titrate pH to 6.9 with HCl and add 1×PBS to a final volume of 1 liter.

1. A method for detecting or diagnosing Fabry disease (FD) in a subject, comprising detecting globotriaosylceramide (Gb3) deposits in biomaterial obtained from said subject, wherein said biomaterial is selected from the group consisting of (i) blood smear prepared from whole blood, (ii) peripheral blood mononuclear cells (PBMCs), and (iii) epithelial cells.

2. The method according to claim 1, wherein said epithelial cells are buccal epithelial cell or bladder epithelial cells.

3. The method according to claim 2, wherein said bladder epithelial cells are preferably present in a urine sample.

4. The method according to claim 1, wherein an increased amount of Gb3 deposit positive cells in said biomaterial as compared to a control is indicative for FD.

5.-6. (canceled)

7. The method according to claim 1, comprising:
a) depositing the biomaterial obtained from said subject to a solid support thereby immobilizing said biomaterial, and
b) detecting Gb3 deposit positive cells in said biomaterial.

8.-10. (canceled)

11. The method according to claim 7, further comprising contacting the immobilized biomaterial with a Gb3 binding agent or a reagent metabolizing Gb3 to a Gb3 metabolic product, thereby allowing for detection of Gb3 deposit positive cells

12. The method according to claim 11, wherein the Gb3 binding agent is a Gb3 specific antibody or a Gb3 natural ligand

13.-14. (canceled)

15. The method according to claim 11, wherein the reagent metabolizing Gb3 to a Gb3 metabolic product is alpha-galactosidase.

16. The method according to claim 12, wherein the Gb3 natural ligand is a shiga toxin.

17. (canceled)

18. A method for treatment monitoring of Fabry disease (FD) in a patient, comprising comparing the amount of globotriaosylceramide (Gb3) deposits positive cells detected in biomaterial obtained from said patient to the amount of Gb3 deposits positive cells detected in biomaterial obtained from said patient at an earlier date, wherein said biomaterial is one of (i) blood smear prepared from whole blood (ii) peripheral blood mononuclear cells (PBMCs) and (iii) epithelial cells, wherein the comparison provides an evaluation of effect of FD treatment.

19.-20. (canceled)

21. The method according to claim 18, wherein a decreased amount of Gb3 deposit positive cells as compared to the amount of Gb3 deposit positive cells detected at an earlier date indicates a positive treatment effect.

22. The method according to claim 18, wherein no change or an increased amount of Gb3 deposit positive cells as compared to the amount of Gb3 deposit positive cells detected at an earlier date indicates no treatment effect.

23.-24. (canceled)

25. The method according to claim 18, further comprising detecting Gb3 deposit positive cells in said biomaterial.

26.-32. (canceled)

33. The method according to claim 18, comprising the steps of:
a) depositing the biomaterial obtained from said patient to a solid support thereby immobilizing said biomaterial, and
b) detecting Gb3 deposit positive cells in said biomaterial

34.-36. (canceled)

37. The method according to claim 33, further comprising contacting the immobilized biomaterial with a Gb3 binding agent or a reagent metabolizing Gb3 to a Gb3 metabolic product, thereby allowing for detection of Gb3 deposit positive cells.

38. The method according to claim 37, wherein the Gb3 binding agent is a Gb3 specific antibody or a Gb3 natural ligand.

39.-40. (canceled)

41. The method according to claim 37, wherein the reagent metabolizing Gb3 to a Gb3 metabolic product is alpha-galactosidase.

42. The method according to claim 38, wherein the natural ligand is a shiga toxin.

43.-53. (canceled)

54. A kit comprising:
a) a first solid support for depositing biomaterial, and
b) a Gb3-binding agent or a reagent metabolizing Gb3 to a Gb3 metabolic product allowing for detection of Gb3 deposits in said biomaterial.

55. (canceled)

56. The kit according to claim 54, wherein the biomaterial is selected from the group consisting of (i) whole blood, (ii) PBMCs, and (iii) epithelial cells.

57.-69. (canceled)
